(19) United States
(12) Patent Application Publication
(10) Pub. No.: US 2026/0240458 A1
(43) Pub. Date: Aug. 20, 2026

(54) WEARABLE ANALYTE MONITORING DEVICE WITH REPLACEABLE MICRONEEDLE ARRAY UNIT

(71) Applicant: Biolinq Incorporated, San Diego, CA (US)

(72) Inventors: Anderson MICU, San Diego, CA (US); Todd NEWHOUSE, San Diego, CA (US); Jared Rylan TANGNEY, Encinitas, CA (US); Richard YANG, Carlsbad, CA (US); David MORELOCK, Escondido, CA (US)

(21) Appl. No.: 19/389,817

(22) Filed: Nov. 14, 2025

Related U.S. Application Data
(63) Continuation of application No. PCT/US2024/029703, filed on May 16, 2024.
(60) Provisional application No. 63/466,973, filed on May 16, 2023.
(60) Provisional application No. 63/547,723, filed on Nov. 8, 2023.

(51) Int. Cl. A61B 5/145; A61B 5/00; A61B 5/1473

(52) U.S. Cl. CPC A61B 5/14532; A61B 5/14514; A61B 5/1473; A61B 5/685

(57) ABSTRACT

Aspects are directed to a wearable analyte monitoring device that includes a microneedle array unit and an electronics module that releasably connects to the microneedle array unit. The microneedle array unit includes a microneedle array including microneedles configured to pierce skin for analyte sensing. The electronics module includes electronic components configured to receive signals from the microneedle array and process the signals to generate analyte measurements. The microneedle array may be affixed to a movable retention arm that is movable through an extended configuration to a released configuration to facilitate inserting the microneedles into the skin. The electronics module is fitted within a cavity of the microneedle array unit after the microneedles are inserted into the skin.

### CROSS-REFERENCE TO RELATED APPLICATIONS

This application claims priority to U.S. Provisional Application No. 63/446,973, filed on May 16, 2023, and U.S. Provisional Application No. 63/547,723, filed on Nov. 8, 2023, the contents of which are hereby incorporated by reference in their entirety.

### TECHNICAL FIELD

This invention relates generally to the field of analyte monitoring, such as continuous glucose monitoring.

### BACKGROUND

Diabetes is a chronic disease in which the body does not produce or properly utilize insulin, a hormone that regulates blood glucose. Insulin may be administered to a diabetic patient to help regulate blood glucose levels, though blood glucose levels must nevertheless be carefully monitored to help ensure that timing and dosage are appropriate. Without proper management of their condition, diabetic patients may suffer from a variety of complications resulting from hyperglycemia (high blood sugar levels) or hypoglycemia (low blood sugar levels).

Blood glucose monitors help diabetic patients manage their condition by measuring blood glucose levels from a sample of blood. For example, a diabetic patient may obtain a blood sample through a fingerstick sampling mechanism, transfer the blood sample to a test strip with suitable reagent(s) that react with the blood sample, and use a blood glucose monitor to analyze the test strip to measure glucose level in that blood sample. However, a patient using this process can typically only measure his or her glucose levels at discrete instances in time, which may fail to capture a hyperglycemia or hypoglycemia condition in a timely manner. Yet a more recent variety of glucose monitor is a continuous glucose monitor (CGM) device, which includes implantable transdermal electrochemical sensors that are used to continuously detect and quantify blood glucose levels by proxy measurement of glucose levels in the subcutaneous interstitial fluid. However, conventional CGM devices also have weaknesses including tissue trauma from insertion and signal latency (e.g., due to the time required for the glucose analyte to diffuse from capillary sources to the sensor). These weaknesses also lead to a number of drawbacks, such as pain experienced by the patient when electrochemical sensors are inserted, and limited accuracy in glucose measurements, particularly when blood glucose levels are changing rapidly. Accordingly, there is a need for a new and improved analyte monitoring system.

### SUMMARY

According to an embodiment, the present disclosure relates to analyte monitoring.

In embodiments, the present disclosure relates to a two-piece wearable analyte monitoring device. The two-piece wearable analyte monitoring device includes a microneedle array unit including a microneedle array and an electronics module. The electronics module is configured to fit within a cavity of the microsensor array unit. The cavity may be provided on a proximal surface of the microneedle array unit. The microneedle array unit further includes an opening formed through a distal surface through which a microneedle array is configured to extend. The microneedle array is coupled to a movable retention arm, the movable retention arm configured to transition between an extended configuration and a released configuration, where the microneedle array extends through the opening when the movable retention arm is in the released configuration. The electronics module includes electronic components configured to receive and process signals from the microneedle array. In some variations, the electronics module provides power to the microneedle array unit.

### BRIEF DESCRIPTION OF THE DRAWINGS

FIG. 1 depicts an illustrative schematic of an analyte monitoring system with a microneedle array.

FIG. 2A depicts an illustrative schematic of an analyte monitoring device.

FIG. 2B depicts an illustrative schematic of microneedle insertion depth in an analyte monitoring device.

FIG. 3A-FIG. 3D depict an upper perspective view, a side view, a bottom view, and an exploded view, respectively, of an analyte monitoring device.

FIG. 4A-FIG. 4E depict a perspective exploded view, a side exploded view, a lower perspective view, a side view, and an upper perspective view, respectively, of a sensor assembly in an analyte monitoring device.

FIG. 4F-FIG. 4H depict a perspective exploded view, a side exploded view, and a side view, respectively, of a sensor assembly in an analyte monitoring device.

FIG. 5A depicts an illustrative schematic of a microneedle array. FIG. 5B depicts an illustrative schematic of a microneedle in the microneedle array depicted in FIG. 5A.

FIG. 6 depicts an illustrative schematic of a microneedle array used for sensing multiple analytes.

FIG. 7A depicts a cross-sectional side view of a columnar microneedle having a tapered distal end. FIGS. 7B and 7C are images depicting perspective and detailed views, respectively, of an embodiment of the microneedle shown in FIG. 7A.

FIG. 8 depicts an illustrative schematic of a columnar microneedle having a tapered distal end.

FIG. 9A and FIG. 9B depict illustrative schematics of a microneedle array and a microneedle, respectively. FIG. 9C-FIG. 9F depict detailed partial views of an illustrative variation of a microneedle.

FIG. 10A and FIG. 10B depict an illustrative variation of a microneedle.

FIG. 11A and FIG. 11B depict illustrative schematics of a microneedle array configuration. FIG. 11C and FIG. 11D depict illustrative schematics of a microneedle array configuration.

FIG. 12A and FIG. 12B depict perspective and orthogonal views, respectively, of an illustrative variation of a die including a microneedle array.

FIG. 13A-FIG. 13D depict a first perspective view, a second perspective view, a side view, and an exploded view of aspects of a two-piece wearable analyte monitoring device.

FIG. 14A and FIG. 14B depict a top perspective view and a bottom perspective view of an electronics module.

FIG. 15A-FIG. 15F depict system block diagrams illustrating aspects of a two-piece wearable analyte monitoring device.

FIG. 16A and FIG. 16B depict side perspective views of a cover and a microneedle array unit.

FIG. 17 depicts a method of applying a two-piece wearable analyte monitoring device.

### DETAILED DESCRIPTION

The term “a” or “an” refers to one or more of that entity, (e.g., can refer to plural referents). As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents

Non-limiting examples of various aspects and variations of the invention are described herein and illustrated in the accompanying drawings.

Aspects of the current subject matter are directed to a two-piece wearable analyte monitoring device with a microneedle array unit and an electronics module that releasably interfaces with and connects to the microneedle array unit. The microneedle array unit includes a microneedle array including a plurality of microneedles configured to pierce skin for analyte sensing within the layers of the skin, as further described herein. The electronics module includes a housing in which electronic components are arranged and configured to receive signals from the microneedle array unit and process the received signals to generate analyte measurements. In some variations, the electronics module is reusable. In some variations, as further described herein, the electronics module provides power to the microneedle array unit.

In some variations, the microneedle array is affixed to a movable retention arm that is movable through an extended configuration to a released configuration. In some variations, a protective cover surrounds a proximal or outwardly exposed surface of the microneedle array unit during storage and transport and also includes features that provide for insertion of the microneedles into the skin of a user for analyte sensing. Once the microneedle array unit is applied to the user and the microneedles are inserted into the skin of the user, the electronics module may be fitted within a cavity of the microneedle array unit.

Before providing additional details regarding aspects of the two-piece wearable analyte monitoring device with the microneedle array unit and the electronics module, the following provides a description of some aspects of an analyte monitoring device that may be incorporated with the two-piece wearable analyte monitoring device with the microneedle array unit and the electronics module described herein. The following descriptions are meant to be exemplary, and aspects related to the described wearable analyte monitoring device consistent with the current subject matter are not limited to the examples described herein.

As generally described herein, an analyte monitoring system may include an analyte monitoring device that is worn by a user and includes one or more sensors for monitoring at least one analyte of a user. The sensors may, for example, include one or more electrodes configured to perform electrochemical detection of at least one analyte. The analyte monitoring device may communicate sensor data to an external computing device for storage, display, and/or analysis of sensor data.

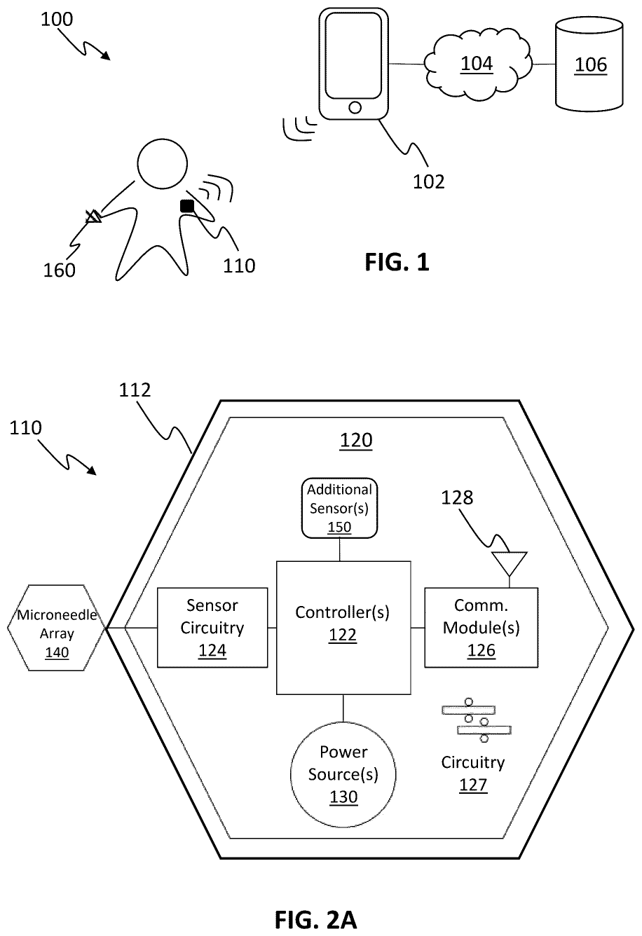

For example, as shown in FIG. 1, an analyte monitoring system 100 may include an analyte monitoring device 110 that is worn by a user, and the analyte monitoring device 110 may be a continuous analyte monitoring device (e.g., continuous glucose monitoring device). The analyte monitoring device 110 may include, for example, a microneedle array comprising at least one electrochemical sensor for detecting and/or measuring one or more analytes in body fluid of a user. The analyte monitoring device 110 may include one or more processors for performing analysis on sensor data, and/or a communication module (e.g., wireless communication module) configured to communicate sensor data to a mobile computing device 102 (e.g., smartphone) or other suitable computing device. In some variations, the mobile computing device 102 may include one or more processors executing a mobile application to handle sensor data (e.g., displaying data, analyzing data for trends, etc.) and/or provide suitable alerts or other notifications related to the sensor data and/or analysis thereof. It should be understood that while in some variations the mobile computing device 102 may perform sensor data analysis locally, other computing device(s) may alternatively or additionally remotely analyze sensor data and/or communicate information related to such analysis with the mobile computing device 102 (or other suitable user interface) for display to the user. Furthermore, in some variations the mobile computing device 102 may be configured to communicate sensor data and/or analysis of the sensor data over a network 104 to one or more storage devices 106 (e.g., server) for archiving data and/or other suitable information related to the user of the analyte monitoring device.

The analyte monitoring devices described herein have characteristics that improve a number of properties that are advantageous for a continuous analyte monitoring device such as a continuous glucose monitoring (CGM) device. For example, the analyte monitoring device described herein have improved sensitivity (amount of sensor signal produced per given concentration of target analyte), improved selectivity (rejection of endogenous and exogenous circulating compounds that can interfere with the detection of the target analyte), and improved stability to help minimize change in sensor response over time through storage and operation of the analyte monitoring device. Additionally, compared to conventional continuous analyte monitoring devices, the analyte monitoring devices described herein have a shorter warm-up time that enables the sensor(s) to quickly provide a stable sensor signal following implantation, as well as a short response time that enables the sensors(s) to quickly provide a stable sensor signal following a change in analyte concentration in the user. Furthermore, as described in further detail below, the analyte monitoring devices described herein may be applied to and function in a variety of wear sites, and provide for pain-free sensor insertion for the user. Other properties such as biocompatibility, sterilizability, and mechanical integrity are also optimized in the analyte monitoring devices described herein.

Although the analyte monitoring systems described herein may be described with reference to monitoring of glucose (e.g., in users with Type 2 diabetes, Type 1 diabetes), it should be understood that such systems may additionally or alternatively be configured to sense and monitor other suitable analytes. As described in further detail below, suitable target analytes for detection may, for example, include glucose, ketones, lactate, and cortisol. One target analyte may be monitored, or multiple target analytes may be simultaneously monitored (e.g., in the same analyte monitoring device). For example, monitoring of other target analytes may enable the monitoring of other indications such as stress (e.g., through detection of rising cortisol and glucose) and ketoacidosis (e.g., through detection of rising ketones).

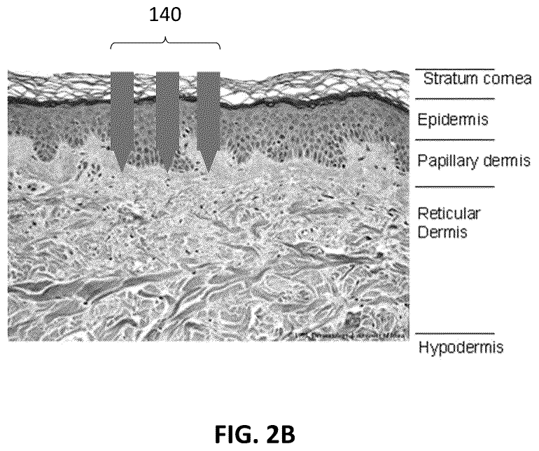

As shown in FIG. 2A, in some variations, an analyte monitoring device 110 may generally include a housing 112 and a microneedle array 140 extending outwardly from the housing. The housing 112, may, for example, be a wearable housing configured to be worn on the skin of a user such that the microneedle array 140 extends at least partially into the skin of the user. For example, the housing 112 may include an adhesive such that the analyte monitoring device 110 is a skin-adhered patch that is simple and straightforward for application to a user. The microneedle array 140 may be configured to puncture the skin of the user and include one or more electrochemical sensors (e.g., electrodes) configured for measuring one or more target analytes that are accessible after the microneedle array 140 punctures the skin of the user. In some variations, the analyte monitoring device 110 may be integrated or self-contained as a single unit, and the unit may be disposable (e.g., used for a period of time and replaced with another instance of the analyte monitoring device 110).

An electronics system 120 may be at least partially arranged in the housing 112 and include various electronic components, such as sensor circuitry 124 configured to perform signal processing (e.g., biasing and readout of electrochemical sensors, converting the analog signals from the electrochemical sensors to digital signals, etc.). The electronics system 120 may also include at least one microcontroller 122 for controlling the analyte monitoring device 110, at least one communication module 126, at least one power source 130, and/or other various suitable passive circuitry 127. The microcontroller 122 may, for example, be configured to interpret digital signals output from the sensor circuitry 124 (e.g., by executing a programmed routine in firmware), perform various suitable algorithms or mathematical transformations (e.g., calibration, etc.), and/or route processed data to and/or from the communication module 124. In some variations, the communication module 126 may include a suitable wireless transceiver (e.g., Bluetooth transceiver or the like) for communicating data with an external computing device 102 via one or more antennas 128. For example, the communication module 126 may be configured to provide uni-directional and/or bi-directional communication of data with an external computing device 102 that is paired with the analyte monitoring device 110. The power source 130 may provide power for the analyte monitoring device 110, such as for the electronics system. The power source 130 may include battery or other suitable source, and may, in some variations, be rechargeable and/or replaceable. Passive circuitry 127 may include various non-powered electrical circuitry (e.g., resistors, capacitors, inductors, etc.) providing interconnections between other electronic components, etc. The passive circuitry 127 may be configured to perform noise reduction, biasing and/or other purposes, for example. In some variations, the electronic components in the electronics system 120 may be arranged on one or more printed circuit boards (PCB), which may be rigid, semi-rigid, or flexible, for example. Additional details of the electronics system 120 are described further below.

In some variations, the analyte monitoring device 110 may further include one or more additional sensors 150 to provide additional information that may be relevant for user monitoring. For example, the analyte monitoring device 110 may further include at least one temperature sensor (e.g., thermistor) configured to measure skin temperature, thereby enabling temperature compensation for the sensor measurements obtained by the microneedle array electrochemical sensors.

The microneedle array 140 in the analyte monitoring device 110 is configured to puncture skin of a user. As shown in FIG. 2B, when the device 110 is worn by the user, the microneedle array 140 may extend into the skin of the user such that electrodes on distal regions of the microneedles rest in the dermis. Specifically, in some variations, the microneedles may be designed to penetrate the skin and access the upper dermal region (e.g., papillary dermis and upper reticular dermis layers) of the skin, in order to enable the electrodes to access interstitial fluid that surrounds the cells in these layers. For example, in some variations, the microneedles may have a height generally ranging between at least 350 μm and about 515 μm. In some variations, one or more microneedles may extend from the housing such that a distal end of the electrode on the microneedle is located less than about 5 mm from a skin-interfacing surface of the housing, less than about 4 mm from the housing, less than about 3 mm from the housing, less than about 2 mm from the housing, or less than about 1 mm from the housing.

In contrast to traditional continuous analyte monitoring devices (e.g., CGM devices), which include sensors typically implanted between about 8 mm and about 10 mm beneath the skin surface in the subcutis or adipose layer of the skin, the analyte monitoring device 110 has a shallower microneedle insertion depth of about 0.25 mm (such that electrodes are implanted in the upper dermal region of the skin) that provides numerous benefits. These benefits include access to dermal interstitial fluid including one or more target analytes for detection, which is advantageous at least because at least some types of analyte measurements of dermal interstitial fluid have been found to closely correlate to those of blood. For example, it has been discovered that glucose measurements performed using electrochemical sensors accessing dermal interstitial fluid are advantageously highly linearly correlated with blood glucose measurements. Accordingly, glucose measurements based on dermal interstitial fluid are highly representative of blood glucose measurements.

Additionally, because of the shallower microneedle insertion depth of the analyte monitoring device 110, a reduced time delay in analyte detection is obtained compared to traditional continuous analyte monitoring devices. Such a shallower insertion depth positions the sensor surfaces in close proximity (e.g., within a few hundred micrometers or less) to the dense and well-perfused capillary bed of the reticular dermis, resulting in a negligible diffusional lag from the capillaries to the sensor surface. Diffusion time is related to diffusion distance according to t=x²/(2D) where t is the diffusion time, x is the diffusion distance, and D is the mass diffusivity of the analyte of interest. Therefore, positioning an analyte sensing element twice as far away from the source of an analyte in a capillary will result in a quadrupling of the diffusional delay time. Accordingly, conventional analyte sensors, which reside in the very poorly vascularized adipose tissue beneath the dermis, result in a significantly greater diffusion distance from the vasculature in the dermis and thus a substantial diffusional latency (e.g., typically 5-20 minutes). In contrast, the shallower microneedle insertion depth of the analyte monitoring device 110 benefits from low diffusional latency from capillaries to the sensor, thereby reducing time delay in analyte detection and providing more accurate results in real-time or near real-time. For example, in some embodiments, diffusional latency may be less than 10 minutes, less than 5 minutes, or less than 3 minutes.

Furthermore, when the microneedle array rests in the upper dermal region, the lower dermis beneath the microneedle array includes very high levels of vascularization and perfusion to support the dermal metabolism, which enables thermoregulation (via vasoconstriction and/or vasodilation) and provides a barrier function to help stabilize the sensing environment around the microneedles. Yet another advantage of the shallower insertion depth is that the upper dermal layers lack pain receptors, thus resulting in a reduced pain sensation when the microneedle array punctures the skin of the user, and providing for a more comfortable, minimally-invasive user experience.

Thus, the analyte monitoring devices and methods described herein enable improved continuous monitoring of one or more target analytes of a user. For example, as described above, the analyte monitoring device may be simple and straightforward to apply, which improves ease-of-use and user compliance. Additionally, analyte measurements of dermal interstitial fluid may provide for highly accurate analyte detection. Furthermore, compared to traditional continuous analyte monitoring devices, insertion of the microneedle array and its sensors may be less invasive and involve less pain for the user. Additional advantages of other aspects of the analyte monitoring devices and methods are further described below.

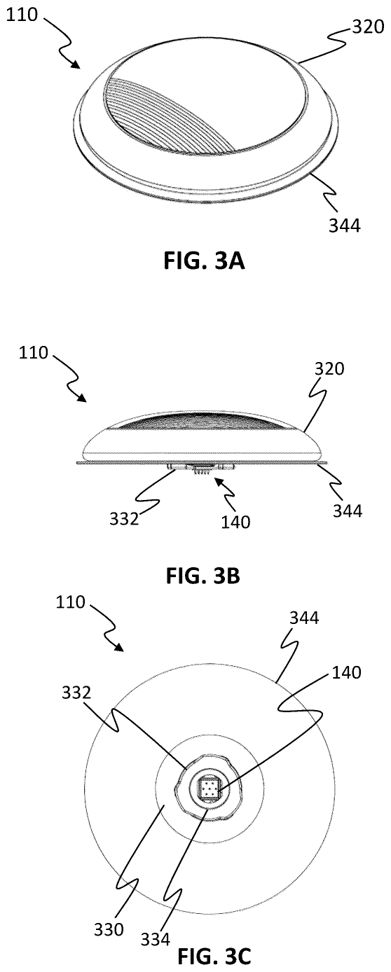

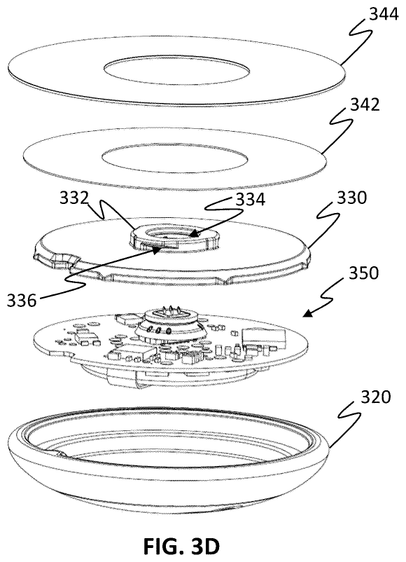

FIG. 3A-FIG. 3D depict aspects of the analyte monitoring device 110. FIG. 3A-FIG. 3D depict an upper perspective view, a side view, a bottom view, and an exploded view, respectively, of the analyte monitoring device 110.

The analyte monitoring device 110 may include a housing that at least partially surrounds or encloses other components (e.g., electronic components) of the analyte monitoring device 110, such as for protection of such components. For example, the housing may be configured to help prevent dust and moisture from entering the analyte monitoring device 110. In some variations, an adhesive layer may attach the housing to a surface (e.g., skin) of a user, while permitting the microneedle array 140 to extend outwardly from the housing and into the skin of the user. Furthermore, in some variations, the housing may generally include rounded edges or corners and/or be low-profile to reduce interference with clothing, etc. worn by the user.

For example, as shown in FIGS. 3A-3D, an example variation of the analyte monitoring device 110 may include a housing cover 320 and a base plate 330, configured to at least partially surround internal components of the analyte monitoring device 110. For example, the housing cover 320 and the base plate 330 may provide an enclosure for a sensor assembly 350 including the microneedle array 140 and electronic components. Once assembled, the microneedle array 140 extends outwardly from a portion of the base plate 330 in a skin-facing direction (e.g., an underside) of the analyte monitoring device 110.

The housing cover 320 and the base plate 330 may, for example, include one or more rigid or semi-rigid protective shell components that may couple together via suitable fasteners (e.g., mechanical fasteners), mechanically interlocking or mating features, and/or an engineering fit. The housing cover 320 and the base plate 330 may include radiused edges and corners and/or other atraumatic features. When coupled together, the housing cover 320 and the base plate 330 may form an internal volume that houses internal components, such as the sensor assembly 350. For example, the internal components arranged in the internal volume may be arranged in a compact, low-profile stack-up as the sensor assembly 350.

The analyte monitoring device 110 may include one or more adhesive layers to attach the analyte monitoring device 110 (e.g., the coupled together housing cover 320 and the base plate 330) to a surface (e.g., the skin) of a user. As shown in FIG. 3D, the one or more adhesive layers may include an inner adhesive layer 342 and an outer adhesive layer 344. The inner adhesive layer 342 may adhere to the base plate 330, and the outer adhesive layer 344 may adhere to the inner adhesive layer 342 and, on its outward facing side, provide an adhesive for adhering (e.g., temporarily) to the skin of the user. The inner adhesive layer 342 and the outer adhesive layer 344 together act as a double-sided adhesive for adhering the analyte monitoring device 110 to the skin of the user. The outer adhesive layer 344 may be protected by a release liner that the user removes to expose the adhesive prior to skin application. In some variations, a single adhesive layer is provided. In some variations, the outer adhesive layer 344 and/or the inner adhesive layer 342 may have a perimeter that extends farther than the perimeter or periphery of the housing cover 320 and the base plate 330. This may increase surface area for attachment and increase stability of retention or attachment to the skin of the user. The inner adhesive layer 342 and the outer adhesive layer 344 each have an opening that permits passage of the outwardly extending microneedle array 140, as further described below. The openings of the inner adhesive layer 342 and the outer adhesive layer 344 may generally align with one another but may, in some variations, differ in size such that one opening is smaller than the other opening. In some variations, the openings are substantially the same size.

The base plate 330 has a first surface (e.g., outwardly exposed surface) opposite a second surface and serves as a support and/or connection structure and as a protective cover for the sensor assembly 350. The base plate 330 is sized and shaped to attach to the housing cover 320. The base plate 330 may be shaped to securely fit within the housing cover 320 such that outer edges of the base plate 330 align with corresponding edges of an opening of the housing 320. The alignment may be such that there is no gap between the outer edges of the base plate 330 and the corresponding edges of the opening of the housing cover 320.

A connection member 332 may be formed in a central or near central region of the first surface of the base plate 330. The connection member 332 has a first surface substantially parallel to the first surface of the base plate 330. Sidewalls extend from edges of the first surface of the connection member 332 to the first surface of the base plate 330. A remaining portion of the first surface of the base plate 330 surrounding the connection member 332 may be flat or substantially flat. One or more connector features may extend outwardly from the sidewalls of the connection member 332 to releasably engage with corresponding connectors of a microneedle enclosure. The first surface and the sidewalls of the connection member 332 define, in part, a cavity. The cavity may be further defined through a portion of the base plate 330 adjacent (e.g., below) the connection member 332. The cavity has an opening, and is accessible, on the second surface of the base plate 330. An aperture 334 is formed through the first surface of the connection member 332. The aperture 334 may be sized and shaped such that the microneedle array 140 fits securely within and extends through the aperture 334. For example, sidewalls of the microneedle array 140 may align with corresponding sidewalls of the aperture 334. In some variations, the aperture 334 may be sized and shaped to correspond with an area surrounding the microneedle array 140. The openings in the inner adhesive layer 342 and the outer adhesive layer 344 are sized such that the connection member 332 extends through the openings without interfering with the inner adhesive layer 342 and the outer adhesive layer 344. For example, the diameter of the opening of the inner adhesive layer 342 and the diameter of the opening of the outer adhesive layer 344 is larger than that of the connection member 332.

Although the housing cover 320 and the base plate 330 depicted in FIGS. 3A-3D are substantially circular with the housing cover 320 having a dome shape, in other variations, the housing cover 320 and the base plate 330 may have any suitable shape. For example, in other variations the housing cover 320 and the base plate 330 may be generally prismatic and have an elliptical, triangular, rectangular, pentagonal, hexagonal, or other suitable shape. The outer adhesive layer 344 may extend outwardly from the housing cover 320 and the base plate 330 to extend beyond the perimeter of the housing cover 320. The outer adhesive layer 344 may be circular, as shown in FIGS. 3A-3D or may have an elliptical, triangular, rectangular, pentagonal, hexagonal, or other suitable shape and need not be the same shape as the housing cover 320 and/or the base plate 330.

In some variations, the analyte monitoring device 110 may provide user status, analyte monitoring device status, and/or other suitable information directly via a user interface (e.g., display, indicator lights, etc. as described below) on the analyte monitoring device 110. Thus, in contrast to analyte monitoring devices that may solely communicate information to a separate peripheral device (e.g., mobile phone, etc.) that in turn communicates the information to a user, in some variations such information may be directly provided by the analyte monitoring device 110.

Accordingly, in some variations, the housing cover 320 may include a user interface, such as an interface to provide information in a visual, audible, and/or tactile manner to provide information regarding user status and/or status of the analyte monitoring device, and/or other suitable information. Examples of user status that may be communicated via the user interface include information representative of analyte measurement in the user (e.g., below a predetermined target analyte measurement threshold or range, within a predetermined target analyte measurement range, above a predetermined target analyte measurement threshold or range, increase or decrease of analyte measurement over time, rate of change of analyte measurement, other information relating to trend of analyte measurements, other suitable alerts associated with analyte measurement, etc.). Examples of analyte monitoring device status that may be communicated via the user interface include device operation mode (e.g., associated with device warm-up state, analyte monitoring state, battery power status such as low battery, etc.), a device error state (e.g., operational error, pressure-induced sensing attenuation, fault, failure mode, etc.), device power status, device life status (e.g., anticipated sensor end-of-life), status of connectivity between device and a mobile computing device, and/or the like.

In some variations, the user interface may by default be in an enabled or “on” state to communicate such information at least whenever the analyte monitoring device 110 is performing analyte measurements or whenever the analyte monitoring device 110 is powered on, thereby helping to ensure that information is continuously available to the user. For example, user interface elements may communicate through a display or indicator light(s) (e.g., as described below) not only alerts to flag user attention or recommend remedial action, but also when user status and/or device status are normal. Accordingly, in some variations, a user is not required to perform an action to initiate a scan to learn their current analyte measurement level(s), and such information may always readily be available to the user. In some variations, however, a user may perform an action to disable the user interface temporarily (e.g., similar to a “snooze” button) such as for a predetermined amount of time (e.g., 30 minutes, 1 hour, 2 hours, etc.) after which the user interface is automatically reenabled, or until a second action is performed to reenable the user interface.

In some variations, the user interface of the housing cover 320 may include a display configured to visually communicate information. The display may, for example, include a display screen (e.g., LCD screen, OLED display, electrophoretic display, electrochromic display, etc.) configured to display alphanumeric text (e.g., numbers, letters, etc.), symbols, and/or suitable graphics to communicate information to the user. For example, the display screen may include a numerical information, textual information, and/or a graphics (e.g., sloped line, arrows, etc.) of information such as user status and/or status of the analyte monitoring device. For example, the display screen may include text or graphical representations of analyte measurement levels, trends, and/or recommendations (e.g., physical activity, reduced dietary intake, etc.).

Indicator light(s) on the display may be illuminated in one or more various manners to communicate different kinds of information. For example, an indicator light may be selectively illuminated on or off to communicate information (e.g., illumination “on” indicates one status, while illumination “off” indicates another status). An indicator light may be illuminated in a selected color or intensity to communicate information (e.g., illumination in a first color or intensity indicates a first status, while illumination in a second color or intensity indicates a second status). An indicator light may be illuminated in a selected temporal pattern to communicate information (e.g., illumination in a first temporal pattern indicates a first status, while illumination in a second temporal pattern indicates a second status). For example, an indicator light may be selectively illuminated in one of a plurality of predetermined temporal patterns that differ in illumination frequency (e.g., repeated illumination at a rapid or slow frequency), regularity (e.g., periodic repeated illumination vs. intermittent illumination), duration of illumination “on” time, duration of illumination “off” time, rate of change in illumination intensity, duty cycle (e.g., ratio of illumination “on” time to illumination “off” time), and/or the like, where each predetermined temporal pattern may indicate a respective status.

In some variations, a display may include multiple indicator lights that may be collectively illuminated in one or more predetermined illumination modes or sequences in accordance with one or more predetermined spatial and/or temporal patterns. For example, in some variations, some or all the indicator lights arranged on a display may be illuminated in synchrony or in sequence to indicate a particular status. Accordingly, the selected subset of indicator lights (e.g., the spatial arrangement of the indicator lights that are illuminated) and/or the manner in which they are illuminated (e.g., illumination order, illumination rate, etc.) may indicate a particular status. In some variations, a plurality of indicator lights may illuminate simultaneously or in sequence to increase the diversity of the color palette. For example, in some variations, red, green, and blue LEDs may be illuminated in rapid succession to create the impression of white light to a user.

In some variations, one or more of the above-described illumination modes may be combined in any suitable manner (e.g., combination of varying color, intensity, brightness, luminosity, contrast, timing, location, etc.) to communicate information.

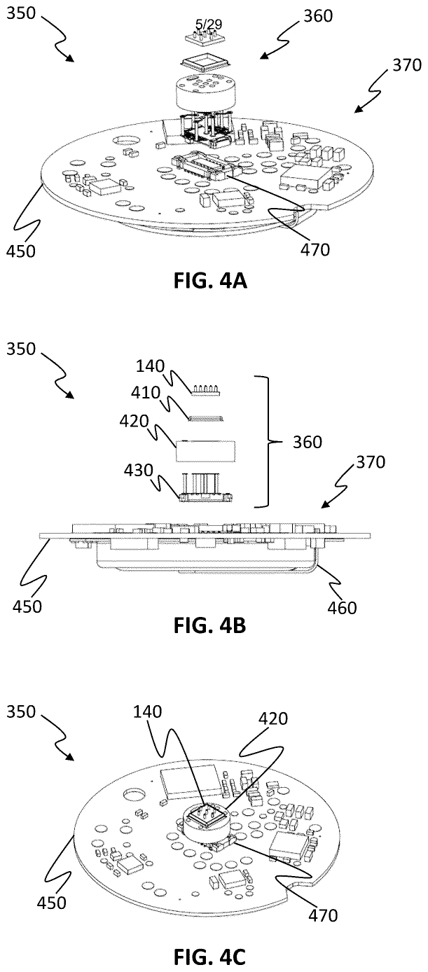

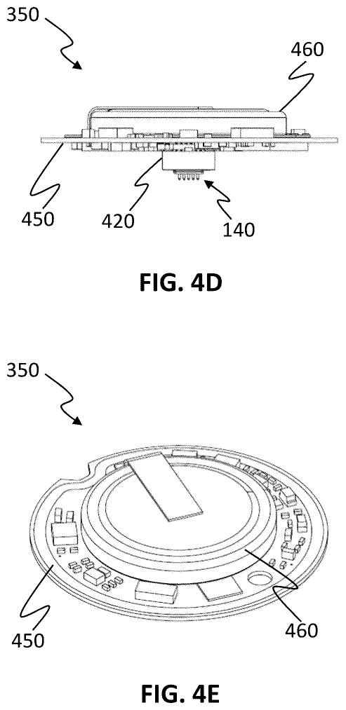

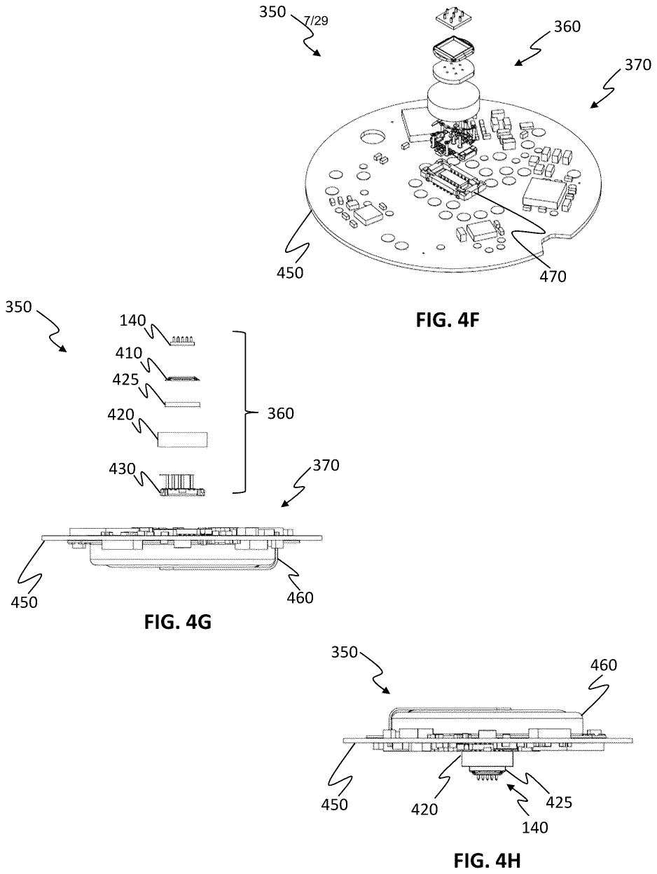

FIGS. 4A-4E depict aspects of the sensor assembly 350 of the analyte monitoring device 110 in a perspective exploded view, a side exploded view, a distal perspective view, a side view, and a proximal perspective view, respectively.

The sensor assembly 350 includes microneedle array components and electronic components to implement analyte detection and processing aspects of the microneedle array-based continuous analyte monitoring device 110 for the detection and measuring of an analyte. In some variations, the sensor assembly 350 is a compact, low-profile stack-up that is at least partially contained within the internal volume defined by the housing cover 320 and the base plate 330.

In some variations, the sensor assembly 350 includes a microneedle array assembly 360 and an electronics assembly 370 that connect to one another to implement the microneedle array analyte detection and processing aspects further described herein. In some variations, the electronics assembly 370 includes a first printed circuit board (PCB) 450 on which electronic components are connected, and the microneedle array assembly 360 includes a second printed circuit board (PCB) 420 on which the microneedle array 140 is connected.

In some variations, the microneedle array assembly 360 includes, in addition to the second PCB 420 and the microneedle array 140, an epoxy skirt 410 and a second PCB connector 430. The microneedle array 140 is coupled to a top side (e.g., outer facing or distal side) of the second PCB 420 so that the individual microneedles of the microneedle array 140 are exposed as described with reference to FIG. 3A-FIG. 3D. The second PCB connector 430 is coupled to a back or proximal side, opposite the top side, of the second PCB 420. The second PCB connector 430 may be an electromechanical connector and may communicatively couple to the first PCB 450 through a first PCB connector 470 on a top side (e.g., outer facing or distal side) of the first PCB 450 to allow for signal communication between the second PCB 420 and the first PCB 450. For example, signals from the microneedle array 140 may be communicated to the first PCB 450 through the second PCB 420, the second PCB connector 430, and the first PCB connector 470.

The second PCB 420 may in part determine the distance to which the microneedle array 140 protrudes from the back plate 330 of the housing. Accordingly, the height of the second PCB 420 may be selected to help ensure that the microneedle array 140 is inserted properly into a user's skin. During microneedle insertion, the first surface (e.g., outer facing surface) of the connection member 332 of the back plate 330 may act as a stop for microneedle insertion. If the second PCB 420 has a reduced height and its top surface is flush or nearly flush with the first surface of the connection member 332, then the connection member 332 may prevent the microneedle array 140 from being fully inserted into the skin.

In some variations, other components (e.g., electronic components such as sensors or other components) may also be connected to the second PCB 420. For example, the second PCB 420 may be sized and shaped to accommodate electronic components on the top side or the back side of the second PCB 420.

In some variations, the epoxy skirt 410 may be deposited along the edges (e.g., the outer perimeter) of the microneedle array 140 to provide a secure fit of the microneedle array 140 within the aperture 334 formed in the connection member 332 of the base plate 330 and/or to relieve the sharp edges along the microneedle array 140, as shown in FIG. 3B and FIG. 3C. For example, the epoxy skirt 410 may occupy portions of the aperture 334 not filled by the microneedle array 140 and/or portions of the cavity defined in the base plate 330 not filled by the second PCB 420. The epoxy skirt 410 may also provide a transition from the edges of the microneedle array 140 to the edge of the second PCB 420. In some variations, the epoxy skirt 410 may be replaced or supplemented by a gasket (e.g., a rubber gasket) or the like.

The electronics assembly 370, having the first PCB 450, includes a battery 460 coupled to a back side of the first PCB 450, opposite the top side on which the first PCB connector 470 is coupled. In some variations, the battery 460 may be coupled on the top side of the first PCB 450 and/or in other arrangements.

FIGS. 4F-4H depict aspects of an alternate variation of the sensor assembly 350 of the analyte monitoring device 110. A perspective exploded view, a side exploded view, and a side view of the sensor assembly 350 are provided, respectively, in FIGS. 4F-4H.

As shown, in the sensor assembly 350, an additional PCB component, an intermediate PCB 425, is incorporated. In some variations, the intermediate PCB 425 is part of the microneedle array assembly 360 and is positioned between and connected to the second PCB 420 and the microneedle array 140. The intermediate PCB 425 may be added to increase the height of the microneedle array assembly 360 such that the microneedle array 140 extends at a further distance from the base plate 330, which may aid in insertion of the microneedle array 140 into the skin of a user. The microneedle array 140 is coupled to a top side (e.g., outer facing side) of the intermediate PCB 425 so that the individual microneedles of the microneedle array 140 are exposed as described with reference to FIG. 3A-FIG. 3D. The second PCB 420 is coupled to a back side, opposite the top side, of the intermediate PCB 425, and the second PCB connector 430 is coupled to a back side, opposite the top side, of the second PCB 420. The epoxy skirt 410 (which may be replaced or supplemented by a gasket of the like) provides a transition from the edges of the microneedle array 140 to the edge of the intermediate PCB 425.

The intermediate PCB 425 with the second PCB 420 in part determine the distance to which the microneedle array 140 protrudes through the aperture 334 of the back plate 330. The incorporation of the intermediate PCB 425 provides an additional height to help ensure that the microneedle array 140 is properly inserted into a user's skin. In some variations, the top side (e.g., outer facing side) of the intermediate PCB 425 extends through and out of the aperture 334 so that the first surface (e.g., top, exposed surface) of the connection member 332 surrounding the aperture 334 does not prevent the microneedle array from being fully inserted into the skin. In some variations, the top side (e.g., outer facing side) of the intermediate PCB 425 does not extend out of the aperture 334 but the increased height (by virtue of incorporating the intermediate PCB 425) ensures that the microneedle array 140 protrudes at a sufficient distance from the back plate 330 of the housing.

In some variations, a microneedle enclosure may be provided for releasable attachment to the analyte monitoring device 110. The microneedle enclosure may provide a protective environment or enclosure in which the microneedle array 140 may be safely contained, thereby ensuring the integrity of the microneedle array 140 during certain stages of manufacture and transport of the analyte monitoring device 110, prior to application of the analyte monitoring device 110. The microneedle enclosure is releasable or removable from the analyte monitoring device 110 to allow for the microneedle array 140 to be exposed and ready for insertion into the skin of the user, as further described herein.

In some variations, the microneedle enclosure, by providing an enclosed and sealed environment in which the microneedle array 140 may be contained, provides an environment in which the microneedle array 140 may be sterilized. For example, the microneedle enclosure with the microneedle array 140 may be subjected to a sterilization process, during which the sterilization penetrates the microneedle enclosure so that the microneedle array 140 is also sterilized. As the microneedle array 140 is contained in an enclosed environment, the microneedle array 140 remains sterilized until removed from the enclosed environment.

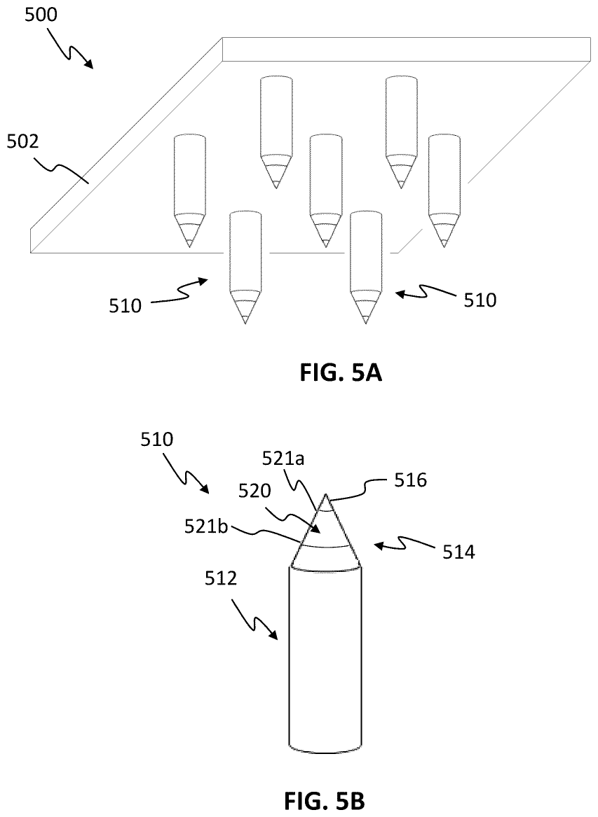

As shown in the schematic of FIG. 5A, in some variations, a microneedle array 510 for use in sensing an analyte may include one or more microneedles 510 projecting from a substrate surface 502. The substrate surface 502 may, for example, be a generally planar semiconductor (e.g. Silicon) substrate and one or more microneedles 510 may project orthogonally from the planar surface. Generally, as shown in FIG. 5B, a microneedle 510 may include a body portion 512 (e.g., shaft) and a tapered distal portion 514 configured to puncture skin of a user. In some variations, the tapered distal portion 514 may terminate in an insulated distal apex 516. The microneedle 510 may further include an electrode 520 on a surface of the tapered distal portion. In some variations, electrode-based measurements may be performed at the interface of the electrode and interstitial fluid located within the body (e.g., on an outer surface of the overall microneedle). In some variations, the microneedle 510 may have a solid core (e.g., solid body portion), though in some variations the microneedle 510 may include one or more lumens, which may be used for drug delivery or sampling of the dermal interstitial fluid, for example. Other microneedle variations, such as those described below, may similarly either include a solid core or one or more lumens.

The microneedle array 500 may be at least partially formed from a semiconductor (e.g., silicon) substrate and include various material layers applied and shaped using various suitable microelectromechanical systems (MEMS) manufacturing techniques (e.g., deposition and etching techniques), as further described below. The microneedle array may be reflow-soldered to a circuit board, similar to a typical integrated circuit. Furthermore, in some variations the microneedle array 500 may include a three electrode setup including a working (sensing) electrode having an electrochemical sensing coating (including a biorecognition element such as an aptamer or an enzyme) that enables detection of the analyte, a reference electrode, and a counter electrode. In other words, the microneedle array 500 may include at least one microneedle 510 that includes a working electrode, at least one microneedle 510 including a reference electrode, and at least one microneedle 510 including a counter electrode. Additional details of these types of electrodes are described in further detail below.

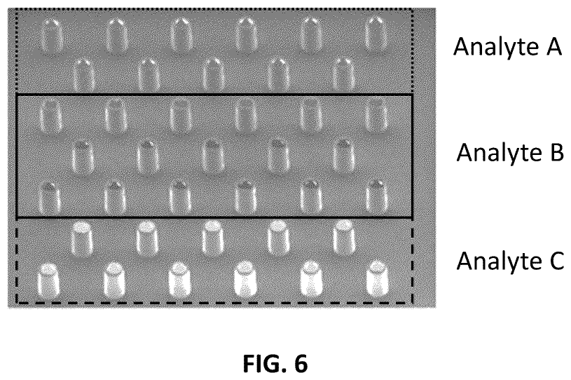

In some variations, the microneedle array 500 may include a plurality of microneedles that are insulated such that the electrode on each microneedle in the plurality of microneedles is individually addressable and electrically isolated from every other electrode on the microneedle array. The resulting individual addressability of the microneedle array 500 may enable greater control over each electrode's function, since each electrode may be separately probed. For example, the microneedle array 500 may be used to provide multiple independent measurements of a given analyte, which improves the device's sensing reliability and accuracy. Furthermore, in some variations the electrodes of multiple microneedles may be electrically connected to produce augmented signal levels. As another example, the same microneedle array 500 may additionally or alternatively be interrogated to simultaneously measure multiple analytes to provide a more comprehensive assessment of physiological status. For example, as shown in the schematic of FIG. 6, a microneedle array may include a portion of microneedles to detect s first analyte A, a second portion of microneedles to detect a second Analyte B, and a third portion of microneedles to detect a third Analyte C. It should be understood that the microneedle array may be configured to detect any suitable number of analytes (e.g., 1, 2, 3, 4, 5 or more, etc.), provided that at least one of the analytes is analyte.

In some variations of microneedles (e.g., microneedles with a working electrode), the electrode 520 may be located proximal to the insulated distal apex 516 of the microneedle. In other words, in some variations the electrode 520 does not cover the apex of the microneedle. Rather, the electrode 520 may be offset from the apex or tip of the microneedle. The electrode 520 being proximal to or offset from the insulated distal apex 516 of the microneedle advantageously provides more accurate sensor measurements. For example, this arrangement prevents concentration of the electric field at the microneedle apex 516 during manufacturing, thereby avoiding non-uniform electro-deposition of sensing chemistry on the electrode surface 520 that would result in faulty sensing. The electrode 520 may be configured to have an annular shape and may comprise a distal edge 521a and a proximal edge 521b.

As another example, placing the electrode 520 offset from the microneedle apex further improves sensing accuracy by reducing undesirable signal artefacts and/or erroneous sensor readings caused by stress upon microneedle insertion. The distal apex of the microneedle is the first region to penetrate into the skin, and thus experiences the most stress caused by the mechanical shear phenomena accompanying the tearing or cutting of the skin. If the electrode 520 were placed on the apex or tip of the microneedle, this mechanical stress may delaminate the electrochemical sensing coating on the electrode surface when the microneedle is inserted, and/or cause a small yet interfering amount of tissue to be transported onto the active sensing portion of the electrode. Thus, placing the electrode 520 sufficiently offset from the microneedle apex may improve sensing accuracy. For example, in some variations, a distal edge 521a of the electrode 520 may be located at least about 10 μm (e.g., between about 20 μm and about 30 μm) from the distal apex or tip of the microneedle, as measured along a longitudinal axis of the microneedle.

The body portion 512 of the microneedle 510 may further include an electrically conductive pathway extending between the electrode 520 and a backside electrode or other electrical contact (e.g., arranged on a backside of the substrate of the microneedle array). The backside electrode may be soldered to a circuit board, enabling electrical communication with the electrode 520 via the conductive pathway. For example, during use, the in-vivo sensing current (inside the dermis) measured at a working electrode is interrogated by the backside electrical contact, and the electrical connection between the backside electrical contact and the working electrode is facilitated by the conductive pathway. In some variations, this conductive pathway may be facilitated by a metal via running through the interior of the microneedle body portion (e.g., shaft) between the microneedle's proximal and distal ends. Alternatively, in some variations the conductive pathway may be provided by the entire body portion being formed of a conductive material (e.g., doped silicon). In some of these variations, the complete substrate on which the microneedle array 500 is built upon may be electrically conductive, and each microneedle 510 in the microneedle array 500 may be electrically isolated from adjacent microneedles 510 as described below. For example, in some variations, each microneedle 510 in the microneedle array 500 may be electrically isolated from adjacent microneedles 510 with an insulative barrier including electrically insulative material (e.g., dielectric material such as silicon dioxide) that surrounds the conductive pathway extending between the electrode 520 and backside electrical contact. For example, body portion 512 may include an insulative material that forms a sheath around the conductive pathway, thereby preventing electrical communication between the conductive pathway and the substrate. Other example variations of structures enabling electrical isolation among microneedles are described in further detail below.

Such electrical isolation among microneedles in the microneedle array permits the sensors to be individually addressable. This individually addressability advantageously enables independent and parallelized measurement among the sensors, as well as dynamic reconfiguration of sensor assignment (e.g., to different analytes). In some variations, the electrodes in the microneedle array can be configured to provide redundant analyte measurements, which is an advantage over conventional analyte monitoring devices. For example, redundancy can improve performance by improving accuracy (e.g., averaging multiple analyte measurement values from different microneedles which reduces the effect of extreme high or low sensor signals on the determination of analyte levels) and/or improving reliability of the device by reducing the likelihood of total failure.

In some variations, as described in further detail below with respective different variations of the microneedle, the microneedle array may be formed at least in part with suitable semiconductor and/or MEMS fabrication techniques and/or mechanical cutting or dicing. Such processes may, for example, be advantageous for enabling large-scale, cost-efficient manufacturing of microneedle arrays.

Described herein are further example variations of microneedle structures incorporating one or more of the above-described microneedle features for a microneedle array in an analyte monitoring device.

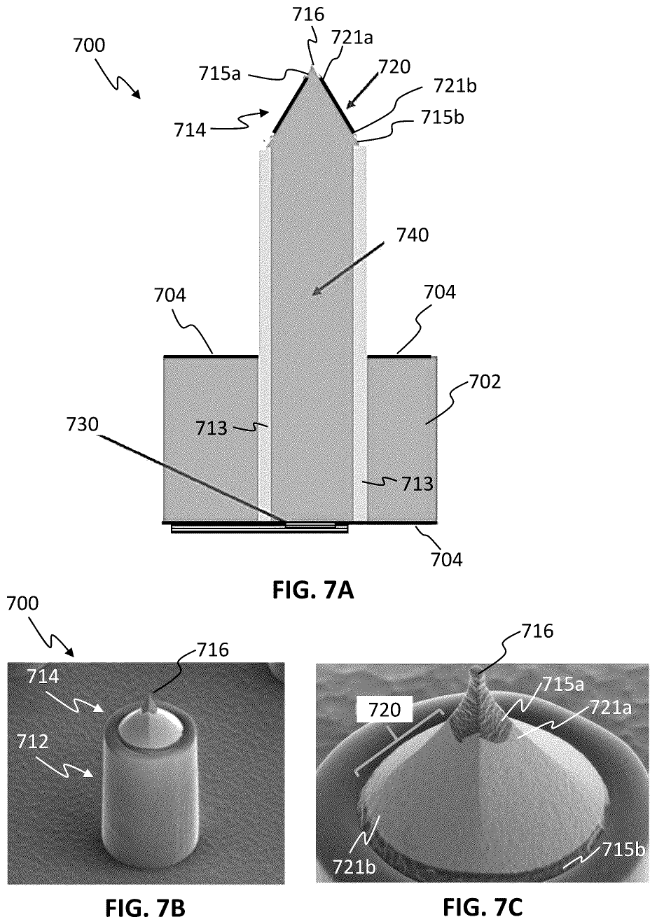

In some variations, a microneedle may have a generally columnar body portion and a tapered distal portion with an electrode. For example, FIGS. 7A-7C illustrate an example variation of a microneedle 700 extending from a substrate 702. FIG. 7A is a side cross-sectional view of a schematic of microneedle 700, while FIG. 7B is a perspective view of the microneedle 700 and FIG. 7C is a detailed perspective view of a distal portion of the microneedle 700. As shown in FIGS. 7B and 7C, the microneedle 700 may include a columnar body portion 712, a tapered distal portion 714 terminating in an insulated distal apex 716, and an annular electrode 720. The annular electrode 720 includes a conductive material (e.g., Pt, Ir, Au, Ti, Cr, Ni, combinations thereof, etc.) arranged on the tapered distal portion 714, such as, for example, on a segment thereof, and comprises a distal edge 721a and a proximal edge 721b. As shown in FIG. 7A, the annular electrode 720 may be proximal to (offset or spaced apart from) the distal apex 716. The annular electrode 720 may be electrically isolated from the distal apex 716 by a distal insulating surface 715a including an insulating material (e.g., SiO2). For example, the distal edge 721a of the annular electrode 720 may be proximate to a proximal edge of the distal insulating surface 715a of the insulated distal apex 716. In some variations, the distal edge 721a of the annular electrode 720 may be proximal to (e.g., just proximal to, adjacent, abutting) a proximal edge of the distal apex 716 (a proximal edge of the distal insulating surface 715a), while in other variations, the distal edge 721a of the annular electrode 720 may be distal to (e.g., just distal to, adjacent) the proximal edge of the insulated distal apex 716 (proximal edge of the distal insulating surface 715a), but may remain proximal to the apex itself. Accordingly, in some variations, the annular electrode 720 may overlie a portion of the distal insulating surface 715a, but may remain proximal to (and offset from) the insulated distal apex itself.

Also as shown in FIG. 7A, the proximal edge 721b of the annular electrode 720 may be distal to, and in some variations, offset or spaced apart from, the columnar body portion 712. In some variations, the proximal edge 721b of the annular electrode 720 may also be electrically isolated from the columnar body portion 712 by a second distal insulating surface 715b comprising an insulating material (e.g., SiO2) at a proximal end or region of the tapered distal portion 714. For example, the proximal edge 721b of the annular electrode 720 may be proximate to a distal edge of the second distal insulating surface 715b. In some variations, the proximal edge 721b of the annular electrode 720 may be proximal to (e.g., just proximal to, adjacent, abutting) a distal edge the second distal insulating surface 715b, while in other variations, the proximal edge 721b of the annular electrode 720 may be distal to (e.g., just distal to, adjacent) the distal edge of the second distal insulating surface 715b, but may remain proximal to the columnar body portion 712. Accordingly, in some variations, the annular electrode 720 may overlie a portion of the second distal insulating surface 715b but may remain proximal to (and offset from) the columnar body portion 712. As shown in FIG. 7A and in some other variations, the annular electrode 720 may be on only a segment of the surface of the tapered distal portion 714, and may or may not extend to the columnar boy portion 712.

The electrode 720 may be in electrical communication with a conductive core 740 (e.g., conductive pathway) passing along the body portion 712 to a backside electrical contact 730 (e.g., made of Ni/Au alloy) or other electrical pad in or on the substrate 702. For example, the body portion 712 may include a conductive core material (e.g., highly doped silicon). As shown in FIG. 7A, in some variations, an insulating moat 713 including an insulating material (e.g., SiO2) may be arranged around (e.g., around the perimeter) of the body portion 712 and extend at least partially through the substrate 702. Accordingly, the insulating moat 713 may, for example, help prevent electrical contact between the conductive core 740 and the surrounding substrate 702. The insulating moat 713 may further extend over the surface of the body portion 712. Upper and/or lower surfaces of the substrate 702 may also include a layer of substrate insulation 704 (e.g., SiO2). Accordingly, the insulation provided by the insulating moat 713 and/or substrate insulation 704 may contribute at least in part to the electrical isolation of the microneedle 700 that enables individual addressability of the microneedle 700 within a microneedle array. Furthermore, in some variations the insulating moat 713 extending over the surface of the body portion 712 may function to increase the mechanical strength of the microneedle 700 structure.

The microneedle 700 may be formed at least in part by suitable MEMS fabrication techniques such as plasma etching, also called dry etching. For example, in some variations, the insulating moat 713 around the body portion 712 of the microneedle may be made by first forming a trench in a silicon substrate by deep reactive ion etching (DRIE) from the backside of the substrate, then filling that trench with a sandwich structure of SiO2/polycrystalline silicon (poly-Si)/SiO2 by low pressure chemical vapor deposition (LPCVD) or other suitable process. In other words, the insulating moat 713 may passivate the surface of the body portion 712 of the microneedle, and continue as a buried feature in the substrate 702 near the proximal portion of the microneedle. By including largely compounds of silicon, the insulating moat 713 may provide good fill and adhesion to the adjoining silicon walls (e.g., of the conductive core 740, substrate 702, etc.). The sandwich structure of the insulating moat 713 may further help provide excellent matching of coefficient of thermal expansion (CTE) with the adjacent silicon, thereby advantageously reducing faults, cracks, and/or other thermally-induced weaknesses in the insulating structure 713.

The tapered distal portion may be fashioned out by an isotropic dry etch from the frontside of the substrate, and the body portion 712 of the microneedle 700 may be formed from DRIE. The frontside metal electrode 720 may be deposited and patterned on the distal portion by specialized lithography (e.g., electron-beam evaporation) that permits metal deposition in the desired annular region for the electrode 720 without coating the distal apex 716. Furthermore, the backside electrical contact 730 of Ni/Au may be deposited by suitable MEMS manufacturing techniques (e.g., sputtering).

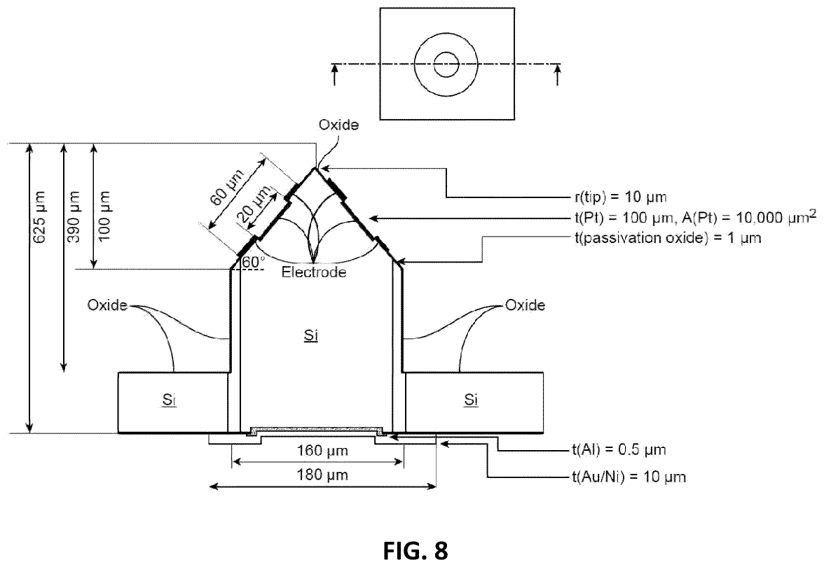

The microneedle 700 may have any suitable dimensions. By way of illustration, the microneedle 700 may, in some variations, have a height of between about 300 μm and about 500 μm. In some variations, the tapered distal portion 714 may have a tip angle between about 60 degrees and about 80 degrees, and an apex diameter of between about 1 μm and about 15 μm. In some variations, the surface area of the annular electrode 720 may include between about 9,000 μm² and about 11,000 μm², or about 10,000 μm². FIG. 8 illustrates various dimensions of an example variation of a columnar microneedle with a tapered distal portion and annular electrode, similar to microneedle 700 described above. As with the microneedle 700 described above, the columnar microneedle of FIG. 8 comprises a columnar body portion, a tapered distal portion terminating in an insulated distal apex, a contact trench formed within the tapered distal portion, and an annular electrode (denoted by “Pt” in FIG. 8) that is arranged on the tapered distal portion and overlays the contact trench. The annular electrode may comprise a conductive material (e.g., Pt, Ir, Au, Ti, Cr, Ni, combinations thereof, etc.). In some variations, the contact trench may have a width of about 1 μm, about 2 μm, about 3 μm, about 4 μm, about 5 μm, about 10 μm, about 15 μm, about 20 μm, about 25 μm, about 30 μm, about 35 μm, about 40 μm, about 45 μm, about 50 μm, or, as shown in FIG. 8, about 20 μm. The annular electrode may comprise a distal edge and a proximal edge, and in some variations, a distance between the distal edge and the proximal edge of the annular electrode may be about 20 μm, about 30 μm, about 40 μm about 50 μm, about 60 μm, about 70 μm, about 80 μm, about 90 μm, about 100 μm, or, as shown in FIG. 8, about 60 μm. In some variations, and as shown in FIG. 8 by the dimensional callouts 60 μm and 20 μm, the annular electrode may overlie the contact trench and, in some instances, a portion of the insulating surfaces (denoted by “Oxide” in FIG. 8) of the tapered distal portion.

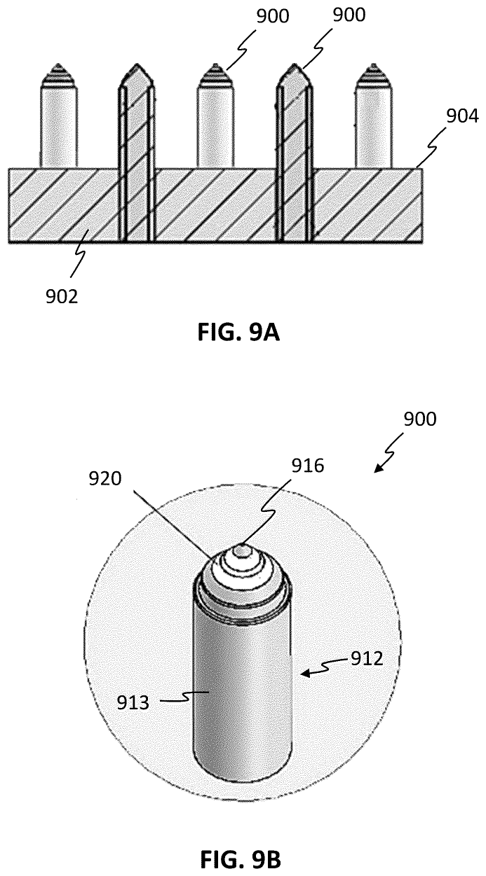

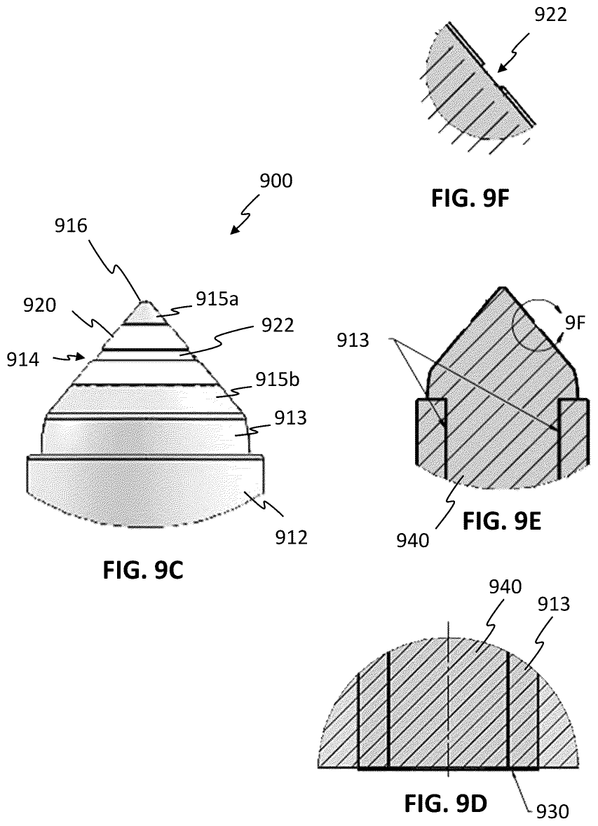

FIGS. 9A-9F illustrate another example variation of a microneedle 900 having a generally columnar body portion extending from a substrate 902 having a top surface 904. The microneedle 900 may be similar to microneedle 700 as described above, except as described below. For example, as shown in FIG. 9B, like the microneedle 700, the microneedle 900 may include a columnar body portion 912, and a tapered distal portion arranged on a cylinder 913 and terminating in an insulated distal apex 916. The cylinder 913 may be insulated and have a smaller diameter than the columnar body portion 912. The microneedle 900 may further include an annular electrode 920 that includes a conductive material and is arranged on the tapered distal portion at a location proximal to (or offset or spaced apart from) the distal apex 916. The electrode 920 may be in electrical communication with a conductive core 940 (e.g., conductive pathway) passing along the body portion 912 to a backside electrical contact 930 (e.g., made of Ni/Au alloy) or other electrical pad in or on the substrate 902. Other elements of microneedle 900 as shown in FIGS. 9A-9F have numbering similar to corresponding elements of microneedle 700.

As can most easily be seen in FIGS. 9B, 9C and 9F, the tapered distal portion 914, and more specifically, the electrode 920 on the tapered distal portion 914 of the microneedle 900, may include a tip contact trench 922. This contact trench may be configured to establish ohmic contact between the electrode 920 and the underlying conductive core 940 of the microneedle. In some variations, the shape of the tip contact trench 922 may include an annular recess formed in the surface of the tapered distal portion 914. In some variations, the shape of the tip contact trench 922 may include an annular recess formed in the surface of the conductive core 940 (e.g., into the body portion of the microneedle, or otherwise in contact with a conductive pathway in the body portion). In some variations, the tip contact trench 922 may be formed in the insulating material on the tapered distal portion 914, and may have a depth about equal to the thickness of the insulating material (e.g., the distal insulating surface 915a and/or the second distal insulating surface 915b). In some instances, the depth of the contact trench may be greater than the thickness of the insulating material such that the contact trench extends beyond a surface of the conductive core 940 (e.g., into the conductive core 940). The electrode 920 may overlie the tip contact trench 922 such that ohmic contact is established between the electrode 920 and the conductive core 940. In some variations, the electrode 920 may extend beyond the tip contact trench 922 such that when the electrode 920 material is deposited onto the conductive core 940, the electrode 920 with the tip contact trench 922 may have a stepped profile when viewed from the side. The tip contact trench 922 may thus advantageously help ensure contact between the electrode 920 and the underlying conductive core 940. Any of the other microneedle variations described herein may also have a similar tip contact trench to help ensure contact between the electrode (which may be, for example, a working electrode, reference electrode, counter electrode, etc.) with a conductive pathway within the microneedle.

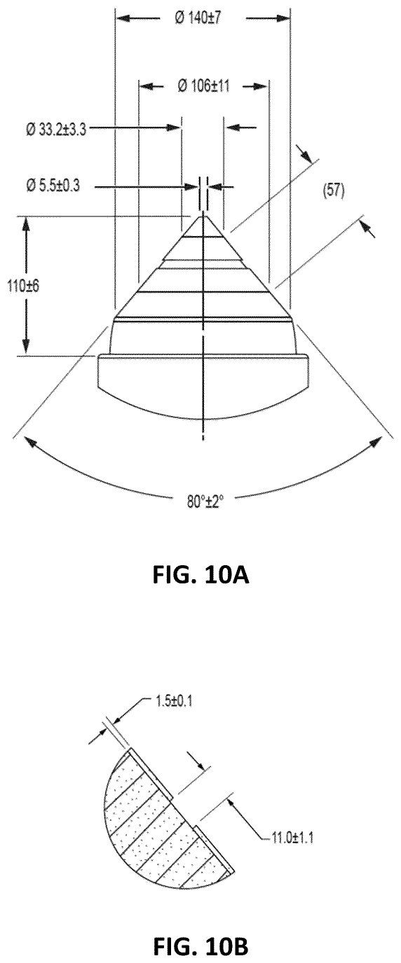

FIGS. 10A and 10B illustrate additional various dimensions of an example variation of a columnar microneedle with a tapered distal portion and annular electrode, similar to microneedle 900 described above. For example, the variation of the microneedle shown in FIGS. 10A and 10B may have a tapered distal portion generally having a taper angle of about 80 degrees (or between about 78 degrees and about 82 degrees, or between about 75 degrees and about 85 degrees), and a cone diameter of about 140 μm (or between about 133 μm and about 147 μm, or between about 130 μm and about 150 μm). The cone of the tapered distal portion may be arranged on a cylinder such that the overall combined height of the cone and cylinder is about 110 μm (or between about 99 μm and about 116 μm, or between about 95 μm and about 120 μm). The annular electrode on the tapered distal portion may have an outer or base diameter of about 106 μm (or between about 95 μm and about 117 μm, or between about 90 μm and about 120 μm), and an inner diameter of about 33.2 μm (or between about 30 μm and about 36 μm, or between about 25 μm and about 40 μm). The length of the annular electrode, as measured along the slope of the tapered distal portion, may be about 57 μm (or between about 55 μm and about 65 μm), and the overall surface area of the electrode may be about 12,700 μm² (or between about 12,500 μm² and about 12,900 μm², or between about 12,000 μm² and about 13,000 μm²). As shown in FIG. 10B, the electrode may furthermore have a tip contact trench extending around a central region of the cone of the tapered distal portion, where the contact may have a width of about 11 μm (or between about 5 μm and about 50 μm, between about 10 μm and about 12 μm, or between about 8 μm and about 14 μm) as measured along the slope of the tapered distal portion, and a trench depth of about 1.5 μm (or between about 0.1 μm and about 5 μm, or between about 0.5 μm and about 1.5 μm, or between about 1.4 μm and about 1.6 μm, or between about 1 μm and about 2 μm). The microneedle has an insulated distal apex having a diameter of about 5.5 μm (or between about 5.3 μm and about 5.8 μm, or between about 5 μm and about 6 μm).

As described above, each microneedle in the microneedle array may include an electrode. In some variations, multiple distinct types of electrodes may be included among the microneedles in the microneedle array. For example, in some variations the microneedle array may function as an electrochemical cell operable in an electrolytic manner with three types of electrodes. In other words, the microneedle array may include at least one working electrode, at least one counter electrode, and at least one reference electrode. Thus, the microneedle array may include three distinct electrode types, though one or more of each electrode type may form a complete system (e.g., the system might include multiple distinct working electrodes). Furthermore, multiple distinct microneedles may be electrically joined to form an effective electrode type (e.g., a single working electrode may be formed from two or more connected microneedles with working electrode sites). Each of these electrode types may include a metallization layer and may include one or more coatings or layers over the metallization layer that help facilitate the function of that electrode.

Generally, the working electrode is the electrode at which oxidation and/or reduction reaction of interest occurs for detection of an analyte of interest. The counter electrode functions to source (provide) or sink (accumulate) the electrons, via an electrical current, that are required to sustain the electrochemical reaction at the working electrode. The reference electrode functions to provide a reference potential for the system; that is, the electrical potential at which the working electrode is biased is referenced to the reference electrode. A fixed, time-varying, or at least controlled potential relationship is established between the working and reference electrodes, and within practical limits no current is sourced from or sinked to the reference electrode. Additionally, to implement such a three-electrode system, the analyte monitoring device may include a suitable potentiostat or electrochemical analog front end to maintain a fixed potential relationship between the working electrode and reference electrode contingents within the electrochemical system (via an electronic feedback mechanism), while permitting the counter electrode to dynamically swing to potentials required to sustain the redox reaction of interest.

Multiple microneedles (e.g., any of the microneedle variations described herein, each of which may have a working electrode, counter electrode, or reference electrode as described above) may be arranged in a microneedle array. Considerations of how to configure the microneedles include factors such as desired insertion force for penetrating skin with the microneedle array, optimization of electrode signal levels and other performance aspects, manufacturing costs and complexity, etc.

For example, the microneedle array may include multiple microneedles that are spaced apart at a predefined pitch (distance between the center of one microneedle to the center of its nearest neighboring microneedle). In some variations, the microneedles may be spaced apart with a sufficient pitch so as to distribute force (e.g., avoid a “bed of nails” effect) that is applied to the skin of the user to cause the microneedle array to penetrate the skin. As pitch increases, force required to insert the microneedle array tends to decrease and depth of penetration tends to increase. However, it has been found that pitch only begins to affect insertion force at low values (e.g., less than about 150 μm). Accordingly, in some variations the microneedles in a microneedle array may have a pitch of at least 200 μm, at least 300 μm, at least 400 μm, at least 500 μm, at least 600 μm, at least 700 μm, or at least 750 μm. For example, the pitch may be between about 200 μm and about 800 μm, between about 300 μm and about 700 μm, or between about 400 μm and about 600 μm. In some variations, the microneedles may be arranged in a periodic grid, and the pitch may be uniform in all directions and across all regions of the microneedle array. Alternatively, the pitch may be different as measured along different axes (e.g., X, Y directions) and/or some regions of the microneedle array may include a smaller pitch while other may include a larger pitch.

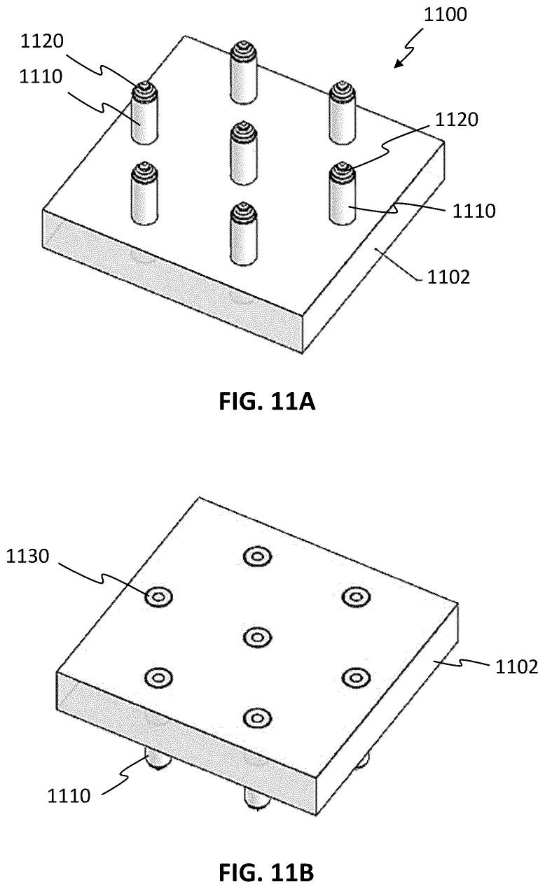

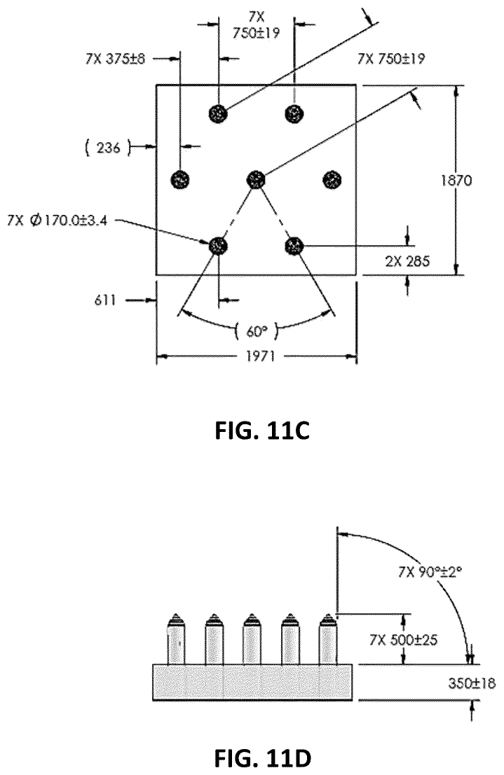

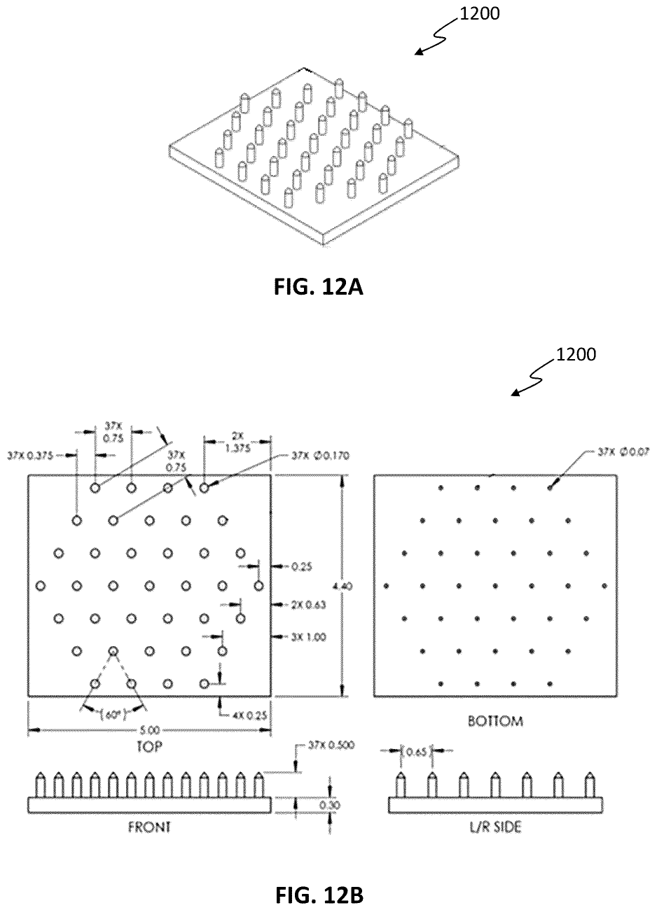

Furthermore, for more consistent penetration and in some variations, microneedles may be spaced equidistant from one another (e.g., same pitch in all directions). To that end, in some variations, the microneedles in a microneedle array may be arranged in a hexagonal configuration as shown in FIGS. 11A-11C and 12A-12B. Alternatively, the microneedles in a microneedle array may arranged in a rectangular array (e.g., square array), or in another suitable symmetrical manner.

FIGS. 11A and 11B depict perspective views of an illustrative schematic of seven microneedles 1110 arranged in an example variation of a microneedle array 1100. The seven microneedles 1110 are arranged in a hexagonal array on a substrate 1102. As shown in FIG. 11A, the electrodes 1120 are arranged on distal portions of the microneedles 1110 extending from a first surface of the substrate 1102. As shown in FIG. 11B, proximal portions of the microneedles 1110 are conductively connected to respective backside electrical contacts 1130 on a second surface of the substrate 1102 opposite the first surface of the substrate 1102. FIGS. 11C and 11D depict plan and side views of an illustrative schematic of a microneedle array similar to microneedle array 1100. As shown in FIGS. 11C and 11D, the seven microneedles are arranged in a hexagonal array with an inter-needle center-to-center pitch of about 750 μm between the center of each microneedle and the center of its immediate neighbor in any direction. In other variations the inter-needle center-to-center pitch may be, for example, between about 700 μm and about 800 μm, or between about 725 μm and about 775 μm. The microneedles may have an approximate outer shaft diameter of about 170 μm (or between about 150 μm and about 190 μm, or between about 125 μm and about 200 μm) and a height of about 500 μm (or between about 475 μm and about 525 μm, or between about 450 μm and about 550 μm).

FIGS. 12A and 12B depict an illustrative schematic of 37 microneedles arranged in an example variation of a microneedle array 1200. The 37 microneedles may, for example, be arranged in a hexagonal array with an inter-needle center-to-center pitch of about 750 μm (or between about 700 μm and about 800 μm, or between about 725 μm and about 775 μm) between the center of each microneedle and the center of its immediate neighbor in any direction. FIG. 12A depicts an illustrative schematic of an example variation of a die including the microneedle arrangement. Example dimensions of the die (e.g., about 4.4 mm by about 5.0 mm) and the microneedle array 1200 are shown in FIG. 12B.

One consideration for determining configuration of a microneedle array may be overall signal level provided by the microneedles. Generally, signal level at each microneedle is invariant of the total number of microneedle elements in an array. However, signal levels can be enhanced by electrically interconnecting multiple microneedles together in an array. For example, an array with a large number of electrically connected microneedles is expected to produce a greater signal intensity (and hence increased accuracy) than one with fewer microneedles. However, a higher number of microneedles on a die will increase die cost (given a constant pitch) and will also require greater force and/or velocity to insert into skin. In contrast, a lower number of microneedles on a die may reduce die cost and enable insertion into the skin with reduced application force and/or velocity. Furthermore, in some variations a lower number of microneedles on a die may reduce the overall footprint area of the die, which may lead to less unwanted localized edema and/or erythema. Accordingly, in some variations, a balance among these factors may be achieved with a microneedle array including 37 microneedles as shown in FIGS. 12A-12B or a microneedle array including seven microneedles as shown in FIGS. 11A-11C. However, in other variations there may be fewer microneedles in an array (e.g., between about 5 and about 35, between about 5 and about 30, between about 5 and about 25, between about 5 and about 20, between about 5 and about 15, between about 5 and about 100, between about 10 and about 30, between about 15 and about 25, etc.) or more microneedles in an array (e.g., more than 37, more than 40, more than 45, etc.).

Additionally, in some variations only a subset of the microneedles in a microneedle array may be active during operation of the analyte monitoring device. For example, a portion of the microneedles in a microneedle array may be inactive (e.g., no signals read from electrodes of inactive microneedles). In some variations, a portion of the microneedles in a microneedle array may be activated at a certain time during operation and remain active for the remainder of the operating lifetime of the device. Furthermore, in some variations, a portion of the microneedles in a microneedle array may additionally or alternatively be deactivated at a certain time during operation and remain inactive for the remainder of the operating lifetime of the device.

In considering characteristics of a die for a microneedle array, die size is a function of the number of microneedles in the microneedle array and the pitch of the microneedles. Manufacturing costs are also a consideration, as a smaller die size will contribute to lower cost since the number of dies that can be formed from a single wafer of a given area will increase. Furthermore, a smaller die size will also be less susceptible to brittle fracture due to the relative fragility of the substrate.

As described above, each microneedle in the microneedle array may include an electrode. In some variations, multiple distinct types of electrodes may be included among the microneedles in the microneedle array. For example, in some variations the microneedle array may function as an electrochemical cell operable in an electrolytic manner with three types of electrodes. In other words, the microneedle array may include at least one working electrode, at least one counter electrode, and at least one reference electrode. Thus, the microneedle array may include three distinct electrode types, though one or more of each electrode type may form a complete system (e.g., the system might include multiple distinct working electrodes). Furthermore, multiple distinct microneedles may be electrically joined to form an effective electrode type (e.g., a single working electrode may be formed from two or more connected microneedles with working electrode sites). Each of these electrode types may include a metallization layer and may include one or more coatings or layers over the metallization layer that help facilitate the function of the particular electrode.

Generally, the working electrode is the electrode at which an oxidation reaction and/or a reduction reaction of interest occurs for detection of an analyte of interest. The counter electrode functions to source (provide) or sink (accumulate) the electrons, via an electrical current, that are required to sustain the electrochemical reaction at the working electrode. The reference electrode functions to provide a reference potential for the system; that is, the electrical potential at which the working electrode is biased is referenced to the reference electrode. A fixed, time-varying, or at least controlled potential relationship is established between the working and reference electrodes, and within practical limits no current is sourced from or sinked to the reference electrode. Additionally, to implement such a three-electrode system, the analyte monitoring device may include a suitable potentiostat or electrochemical analog front end to maintain a fixed potential relationship between the working electrode and reference electrode contingents within the electrochemical system (via an electronic feedback mechanism), while permitting the counter electrode to dynamically swing to potentials required to sustain the redox reaction of interest.

Furthermore, the microneedle arrays described herein may have a high degree of configurability concerning where the working electrode(s), counter electrode(s), and reference electrode(s) are located within the microneedle array. This configurability may be facilitated by the electronics system. Microneedle configurations may include different numbers and/or distributions of working electrodes, counter electrodes, and reference electrodes, and different numbers and/or distributions of active electrodes and inactive electrodes.

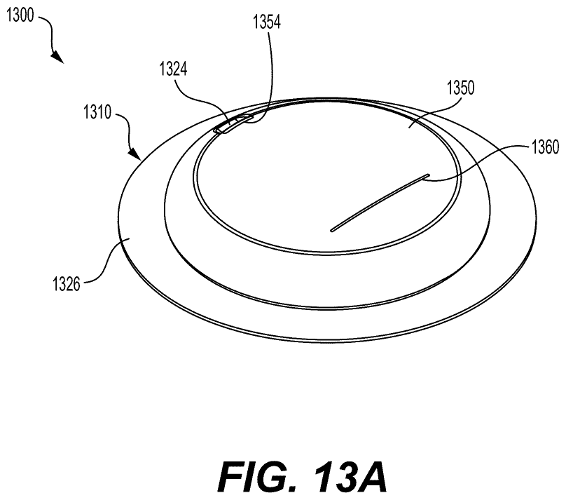

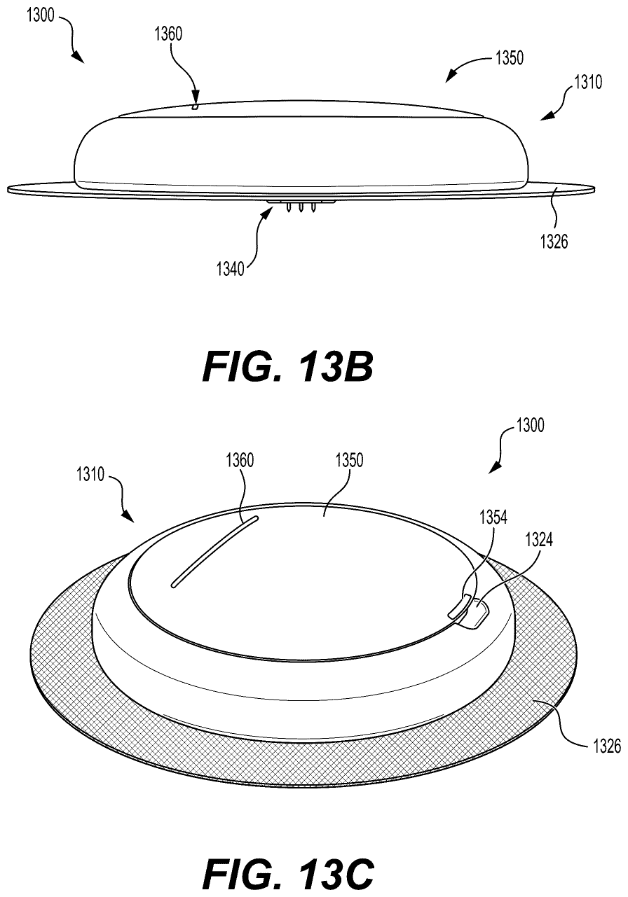

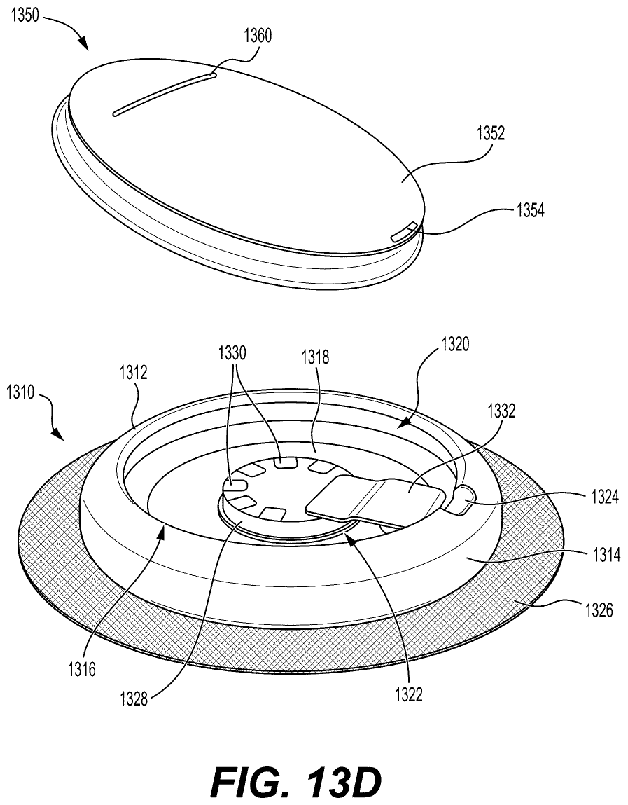

FIGS. 13A-13D depict a first perspective view, a side view, a second perspective view, and an exploded view of a two-piece wearable analyte monitoring device 1300. While the analyte monitoring device 1300 has features described further herein, the wearable analyte monitoring device 1300 may include aspects of the analyte monitoring system described above.

The wearable analyte monitoring device 1300 is a two piece device that includes a microneedle array unit 1310 and an electronics module 1350 that releasably interfaces with and connects to the microneedle array unit 1310. In some variations, the microneedle array unit 1310 is a disposable component, and the electronics module 1350 is a reusable component that may be reused with one or more other microneedle array units. In some variations, the microneedle array unit 1310 includes components necessary for analyte measuring and monitoring (e.g., obtaining analyte signals), and the electronics module 1350 includes durable components that may be reused and/or that last for longer periods of time compared to the microneedle array components that are used for analyte sensing (e.g., the microneedle array). For example, in some variations, the electronics module 1350 may include a power source, a microcontroller or other processing unit, one or more peripheral sensors, one or more output devices, and wireless communication circuitry for communicating with one or more remote devices. The microneedle array unit 1310 may include wireless communication circuitry that enables communication with the electronics module 1350. In some variations, the physical connectors may be provided to establish connection between the microneedle array unit 1310 and the electronics module 1350 for data and/or power transfer.

In some variations, the wireless communication circuitry of the electronics module 1350 is a power source to the microneedle array unit 1310 in addition to providing data to and from the microneedle array unit 1310, as further described below.

In some variations, the microneedle array unit 1310 includes a microcontroller that is configured to receive and process analyte signals from a microneedle array. In some variations, additional processing of the analyte signals is done by the microcontroller of the electronics module 1350.

The microneedle array unit 1310 may include a power source, which may be in addition to or in replacement of the power source of the electronics module 1350. The power source of the microneedle array unit 1310 may be incorporated to provide power to the microneedle array unit 1310 before coupling with the electronics module 1350, in the event of a failure of the power source of the electronics module 1350, and/or to provide power to both the microneedle array unit 1310 and the electronics module 1350. In some variations, the power source of the electronics module 1350 and/or the power source of the microneedle array unit 1310 may be rechargeable and/or replaceable. In situations in which the power source is being recharged or replaced, the power source of the other module may provide power to one or both modules (e.g., the microneedle array unit 1310 and the electronics module 1350). In some variations, each of the microneedle array unit 1310 and the electronics module 1350 may have its own power source.

In some variations, the connection between the microneedle array unit 1310 and the electronics module 1350 is a mechanical connection in which one or more engagement features of the microneedle array unit 1310 and one or more engagement features of the electronics module 1350 engage one another to achieve a secure connection between the microneedle array unit 1310 and the electronics module 1350. In this variation, data and power may be transferred wirelessly. In another variation, electrical contacts may be included in each of the microneedle array unit 1310 and the electronics module 1350, enabling power, from one or more of the microneedle array unit 1310 and the electronics module 1350, to be transferred between the electrical contacts.

FIGS. 13A, 13B, and 13C depict the electronics module 1350 fitted within a cavity 1320 of the microneedle array unit 1310. FIG. 13D depicts the electronics module 1350 disengaged from the microneedle array unit 1310.

Referring to FIG. 13D, the microneedle array unit 1310 includes a base 1312 having a body that includes sidewalls 1314, a proximal opening 1316, and a distal surface 1318 opposite the proximal opening 1316. A cavity 1320 is defined by the sidewalls 1314, the proximal opening 1316, and the distal surface 1318. The distal surface 1318 is generally a flat, planar surface, although in some variations the distal surface 1318 may include curvatures or other features on its distal side that facilitate connection to an adhesive or conformance to a skin surface of a user. A distal opening 1322 is formed through a portion of the distal surface 1318.

The cavity 1320 is sized and shaped to receive and securely hold the electronics module 1350, which includes an electronics housing 1352 in which various components, further described herein, may be arranged. In some variations, inner sidewalls of the cavity 1320 generally correspond to outer sidewalls of the electronics housing 1352, and the span of the cavity 1320 may be slightly larger than that of the electronics housing 1352 such that the electronics housing 1352 fits securely within the cavity 1320. When the electronics housing 1352 is fitted within the cavity 1320 of the microneedle array unit 1310, an interface between the sidewalls 1314 of the base 1312 and the electronics housing 1352 may be a seamless and/or smooth interface.

In some variations, the outer sidewalls of the electronics housing 1352 and/or the inner sidewalls of the cavity 1320 include one or more engagement features that ensure a secure fit therebetween. For example, one or more compliant features or surfaces may be provided. The compliant features or surfaces may provide a snap-fit or otherwise secure engagement of the electronics housing 1352 within the cavity 1320.

In some variations, the microneedle array unit 1310 and the electronics module 1350 have alignment features that serve as a guide to ensure that the electronics housing 1352 is properly aligned within the cavity 1320 of the microneedle array unit 1310. For example, as shown in FIGS. 13A, 13C, and 13D, a notch 1324 may be formed along a portion of the proximal opening 1316 of the base 1312 of the microneedle array unit 1310. The notch 1324 may be a cutout region or groove formed along a periphery of the proximal opening 1316 and/or in a proximal surface of the sidewalls 1314 of the base 1312 and may align and correspond with an indicator mark 1354 formed at an outer edge of a proximal surface of the electronics housing 1352 of the electronics module 1350. In some variations, the indicator mark 1354 is a physical protrusion or ridge. In some variations, the indicator mark 1354 is a line or marking. In some variations, the indicator mark 1354 is a light, such as a light emitting diode or the like, that is configured to illuminate. The length or span of the notch 1324 may correspond to the length or span of the indicator mark 1354. As shown in FIG. 13A and FIG. 13C, when the electronics housing 1352 is fitted within the cavity 1320, the notch 1324 and the indicator mark 1354 align to indicate proper placement of the electronics housing 1352. The notch 1324 may also allow for or facilitate removal of the electronics housing 1352 from the cavity 1320. For example, the notch 1324 provides an opening or groove that allows for a user to remove the electronics housing 1352 from the cavity 1320.

As shown in the side view of the wearable analyte monitoring device 1300 in FIG. 13B, a microneedle array 1340 extends from an underside of the distal surface 1318 of the base 1312 of the microneedle array unit 1310. The microneedle array 1340 extends through the distal opening 1322 formed in the distal surface 1318 of the base 1312 and is surrounded by an adhesive layer 1326. The adhesive layer 1326 may include one or more adhesive layers coupled to an underside or distal side of the microneedle array unit 1310 for adhering the wearable analyte monitoring device 1300 to a skin surface of a user. The wearable analyte monitoring device 1300 is applied to the skin of a user such that the microneedles of the microneedle array 1340 penetrate the skin and the microneedle's electrodes are positioned in the upper dermis for access to dermal interstitial fluid. For example, in some variations, the microneedle array 1340 may be geometrically configured such that the microneedles of the microneedle array 1340 penetrate the outer layer of the skin, the stratum corneum, bore through the epidermis, and come to rest within the papillary or upper reticular dermis. The sensing region, confined to the electrode at the distal extent of each microneedle of the array (as described above) may be configured to rest and remain seated in the papillary or upper reticular dermis following application in order to ensure adequate exposure to circulating dermal interstitial fluid (ISF) without the risk of bleeding or undue influence with nerve endings. The adhesive layer 1326 is configured to adhere to the skin and fix (e.g., secure) the microneedle array 1340 in position.

With further reference to FIG. 13D, details of a first printed circuit board (PCB) 1328 are depicted. A proximal surface of the first PCB 1328 is shown in FIG. 13D. The first PCB 1328 is contained within the cavity 1320 and serves to provide a connection between the microneedle array 1340 and the electronics module 1350, as further described herein. The microneedle array 1340 (not shown in FIG. 13D) is connected to a distal surface of the first PCB 1328. The first PCB 1328 may overlay the distal opening 1322 formed in the distal surface 1318 of the microneedle array unit 1310. In some variations, the first PCB 1328 may generally align with the distal opening 1322 or have a smaller diameter than that of the distal opening 1322. The first PCB 1328 may partially extend through the distal opening 1322. In some variations, the shape of the distal opening 1322 and that of the first PCB 1328 are generally the same, while in other variations, the distal opening 1322 and the first PCB 1328 may be different shapes from one another. In some variations, a gasket or a seal is provided around the distal opening 1322 to provide a tight seal between the distal side of the microneedle array unit 1310 and the cavity 1320 and to prevent moisture ingress into the cavity 1320. The first PCB 1328 may include a stacked PCB arrangement (e.g., two or more PCBs stacked to one another).

A plurality of contact pads 1330 may be provided on the proximal surface of the first PCB 1328 to provide a conductive pathway from the microneedle array 1340 to the electronics module 1350. In some variations, the microneedle array 1340, the first PCB 1328, and the contact pads 1330 may include aspects of the microneedle array assembly 360 described above.

As further shown in FIG. 13D, the first PCB 1328 is affixed or otherwise connected to a movable retention arm 1332 (e.g., a spring-loaded arm, a leaf spring, or the like) that is coupled within the cavity 1320 of the microneedle array unit 1310. The movable retention arm 1332 may transition between different levels of extension. For example, the movable retention arm 1332 may move from an extended (e.g., stressed or stretched) configuration to a released (e.g., relaxed) configuration to assist in placement of the microneedle array 1340 (e.g., penetration of the microneedles of the microneedle array 1340 into the skin of the user) as further described below. In some variations, the microneedle array 1340 may be directly coupled to the movable retention arm 1332 without the need for including the first PCB 1328. The first PCB 1328 or the microneedle array 1340 may be coupled at a proximal side to a distal side of the movable retention arm 1332. Other coupling locations between the first PCB 1328 (or the microneedle array 1340) and the movable retention arm 1332 may be utilized

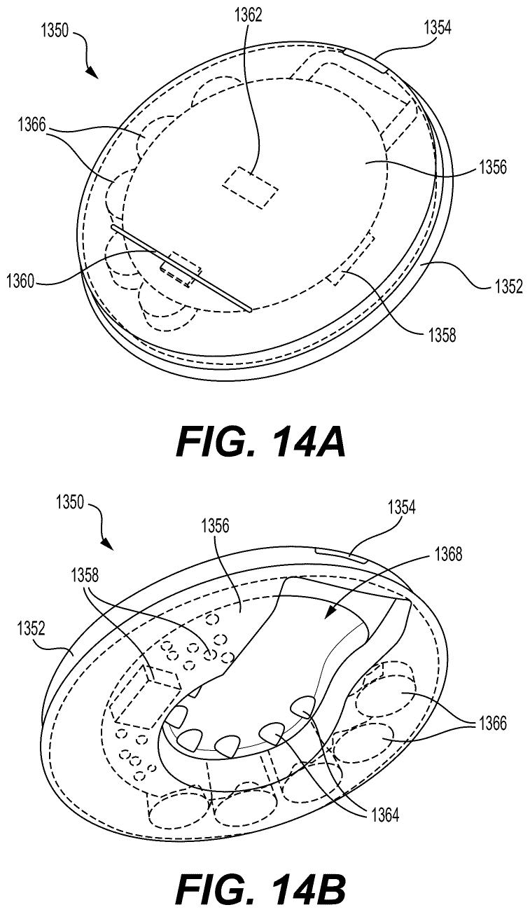

FIG. 14A and FIG. 14B depict a top perspective view and a bottom perspective view of an example variation of the electronics module 1350. The electronics housing 1352 defines an interior space in which various components are positioned. For example, as shown, a second PCB 1356 is positioned within the electronics housing 1352. Electronic and circuitry components 1358 are connected to the second PCB 1356. The electronic and circuitry components 1358 are configured to receive and process signals from the microneedle array 1340 to generate and store analyte measurement data. The electronic and circuitry components 1358 may include one or more of the components described below that implement the microneedle array analyte detection and processing aspects further described herein. For example, the electronic and circuitry components 1358 may include a microcontroller and/or other processing components.

In some variations, the electronics module 1350 may further include one or more of an output device 1360, communication circuitry 1362, which may include one or more antennae, a plurality of connector pins 1364, and one or more batteries 1366. The output device 1360 may be one or more light emitting diodes or the like that are configured to illuminate to provide indications related to analyte detection (e.g., a level of an analyte and/or a rate of change of an analyte measurement), device operation, device status, and/or data from one or more other data sources. In some variations, the output device 1360 may include a plurality of light emitting diodes. The output device 1360 may be a display that outputs graphics related to analyte detection, device operation, device status, and/or data from one or more other data sources.

In some variations, the communication circuitry 1362 and/or the electronic and circuitry components 1358 includes location tracking circuitry (such as a global positioning system (GPS) chip or circuitry) to enable location tracking of the wearable analyte monitoring device 1300. In some variations, coordinates or other location data may be obtained by the location tracking circuitry and communicated to a remote device (e.g., a mobile device and/or a server) that is in communication and/or paired with the wearable analyte monitoring device 1300 through the communication circuitry 1362. In some variations, the location tracking circuitry directly communicates with the remote device. A user may access the remote device (e.g., through an application such as a web-based or mobile application) to initiate a location tracking process to locate the wearable analyte monitoring device 1300. As the location tracking circuitry is part of the electronics module 1350, the location tracking circuitry may be used to track the location of the electronics module 1350 when removed from the microneedle array unit 1310, enabling tracking and/or locating of the separate module. The remote device may send a signal to the electronics module 1350, requesting that the location tracking circuitry obtain and transmit location data. The location data may then be displayed or accessed via the remote device. In another variation, the remote device may issue an alert when the remote device is no longer within a predefined range with the electronics module 1350 and/or the wearable analyte monitoring device 1300.

In some variations, the plurality of connector pins 1364 are positioned on a distal surface of the second PCB 1356 and correspond to and are aligned with the plurality of contact pads 1330 on the proximal surface of the first PCB 1328. When the connector pins 1364 and the contact pads 1330 are connected to or in contact with one another (e.g., each connector pin 1364 has a respective contact pad 1330 to which a connection is formed when the electronics module 1350 is fitted within the cavity 1320 of the microneedle array unit 1310), a connection is made between the microneedle array 1340 and the electronics module 1350 by providing a connection between the first PCB 1328 and the second PCB 1356. The connection may provide for transfer of power and/or data between the electronics module 1350 and the microneedle array unit 1310.

The electronics housing 1352 of the electronics module 1350 is sized and shaped to correspond to the cavity 1320 of the base of the microneedle array unit 1310. The electronics housing 1352 may be generally disc-shaped with a cut-out region 1368 on the distal side, as shown in FIG. 14B. The cut-out region 1368 exposes a portion of the second PCB 1356 to allow for the connection of the second PCB 1356 with the first PCB 1328. The cut-out region 1368 is sized and shaped to accommodate the first PCB 1328 and the movable retention arm 1332 when the electronics module 1350 is fitted within the cavity 1320. For example, the cut-out region 1368 may generally correspond in shape to the first PCB 1328 and the movable retention arm 1332 but have a slightly larger size to allow for the first PCB 1328 and the movable retention arm 1332 to fit therein. A tight seal is provided around the perimeter of the cut-out region 1368 to prevent moisture into the electronics housing 1352.

In some variations, the electronics module 1350 includes a plurality of batteries 1366. The plurality of batteries 1366 may provide enough power such that the electronics module 1350 has battery power sufficient for an extended period of time. The electronics module 1350 may be reusable with various ones of the microneedle array unit 1310. For example, the microneedle array unit 1310 may last for a specified wear time. After the specified wear time, a new microneedle array unit 1310 may be applied, and the electronics module 1350 may be reused with the new microneedle array unit 1310.

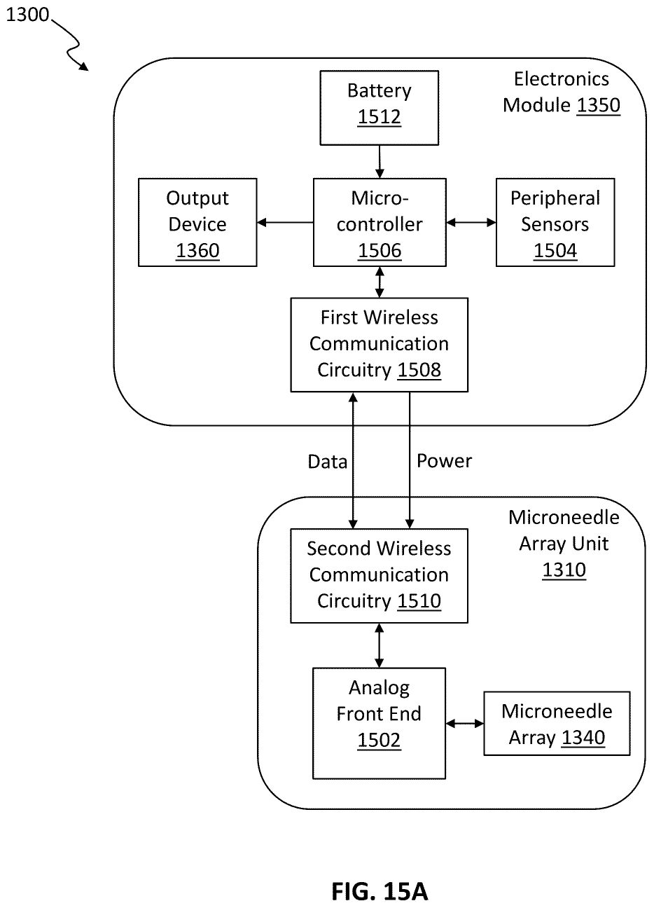

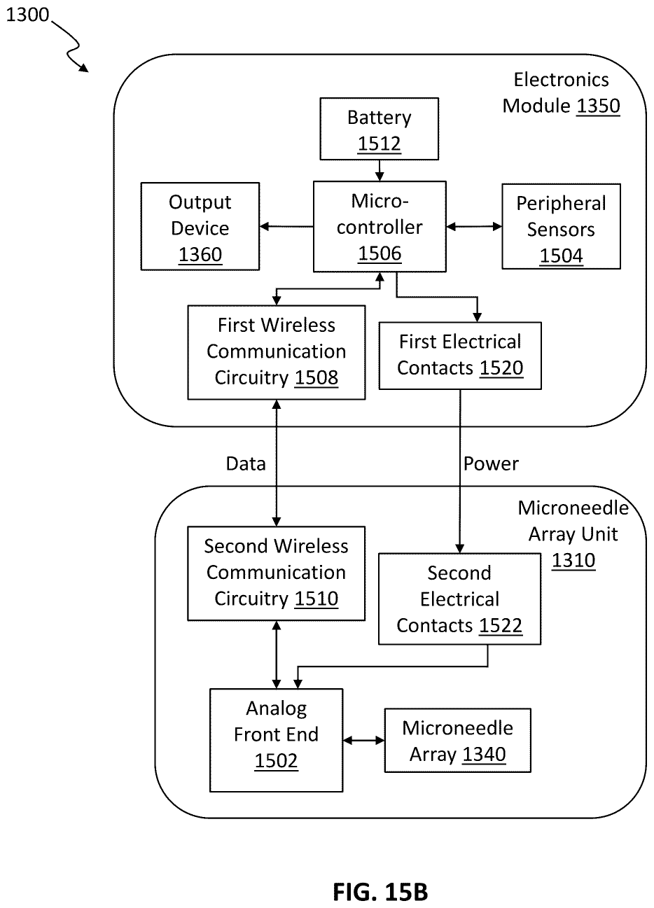

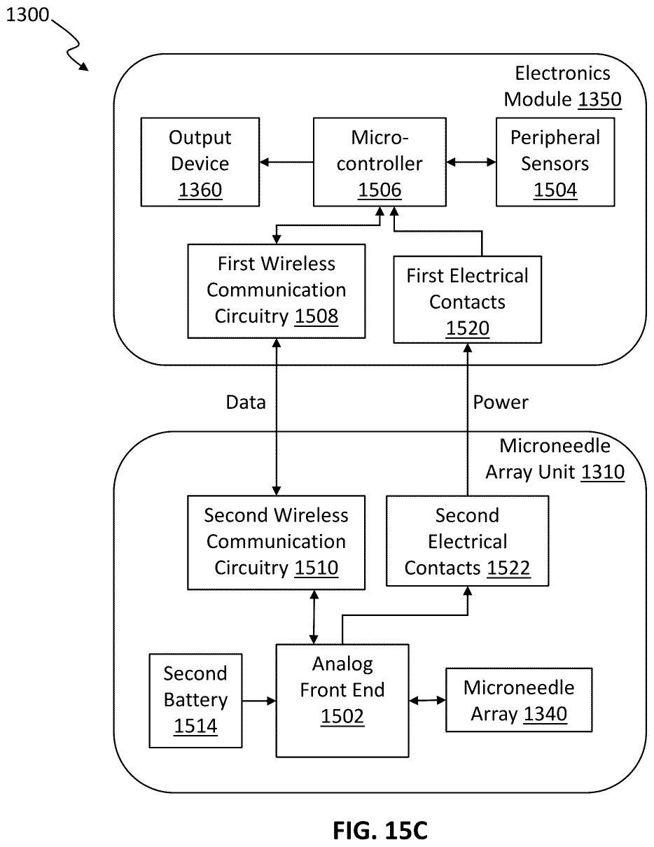

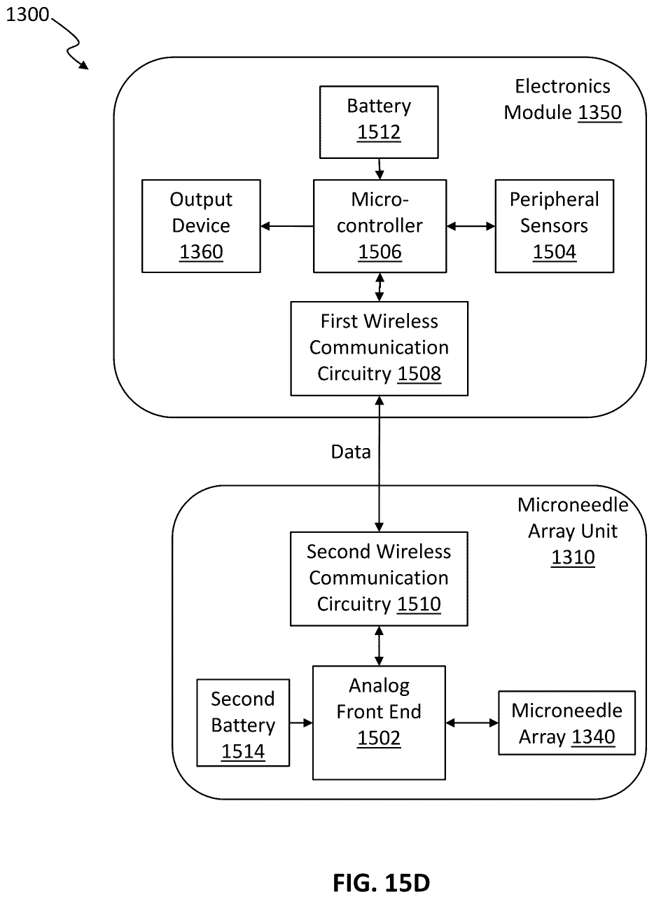

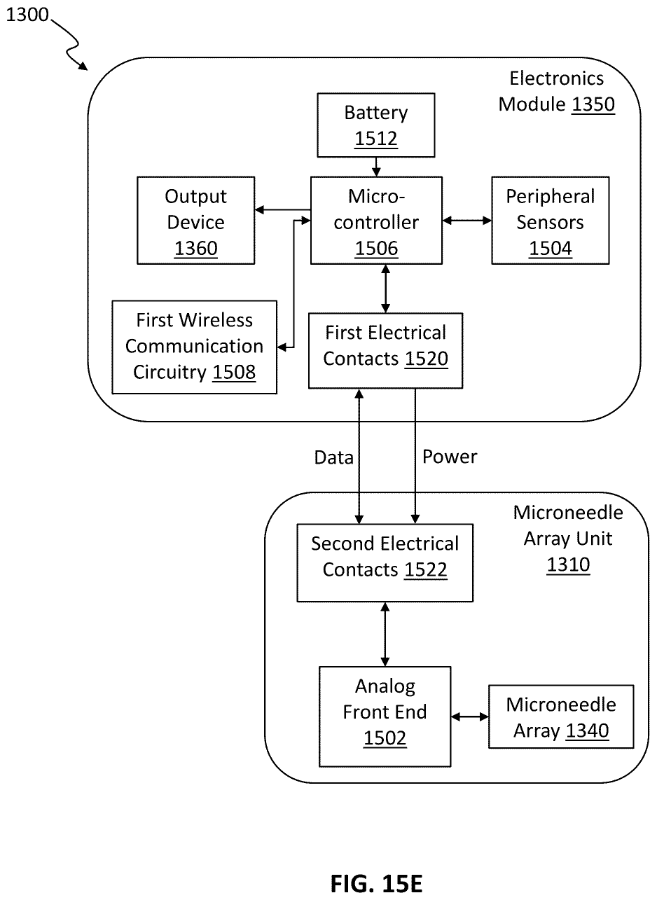

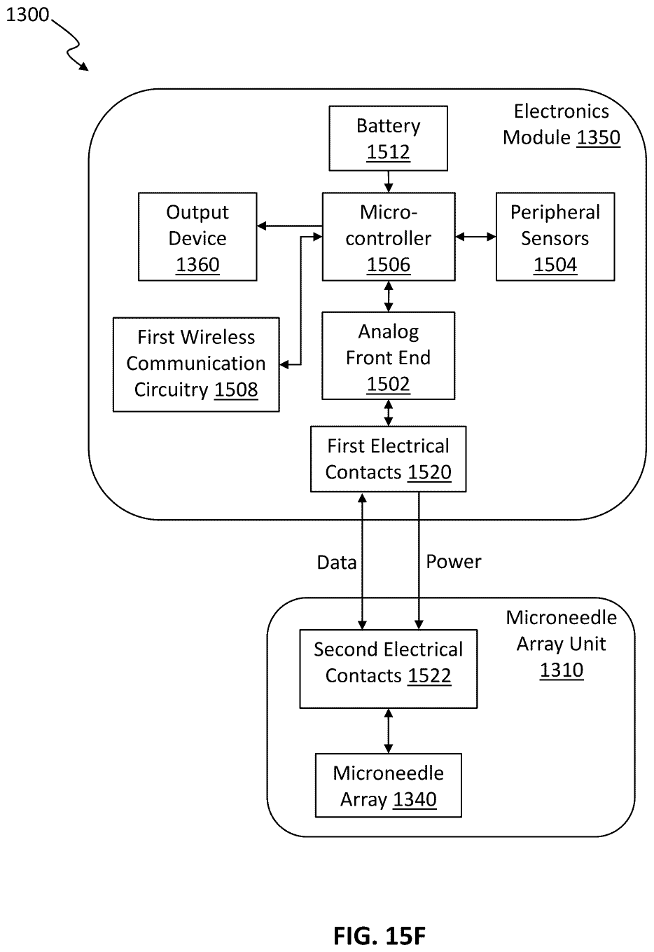

FIG. 15A-FIG. 15F depict system block diagrams illustrating aspects of various implementations of a two-piece wearable analyte monitoring device 1300 including the microneedle array unit 1310 and the electronics module 1350. The microneedle array unit 1310 and the electronics module 1350 may include aspects of the microneedle array assembly 360 and the electronics assembly 370, which may include aspects of the electronics system 120 shown in and described with reference to FIG. 2A.

The electronics module 1350 and the microneedle array unit 1310 may each include one or more respective printed circuit boards (PCBs). In some variations, such as that described with reference to FIG. 14A and FIG. 14B, the PCBs may be connected to establish a connection between the electronics module 1350 and the microneedle array unit 1310, by way of connectors. For example, various connectors and/or contacts may be included to establish connection. Electromechanical connectors may provide for communicative coupling between PCBs to allow for signal communication therebetween. However, in other variations, power and/or data may be transmitted wirelessly between the electronics module 1350 and the microneedle array unit 1310, alleviating the need for electromechanical connection between the two PCBs. In some cases, one of power or data may be transmitted wirelessly while the other is transmitted via physical connectors.

The electronics module 1350 and the microneedle array unit 1310 may include various electronic components to receive and process the electrochemical signals received from the microneedle array 1340, and some electronic components may be included for additional functionality. For example, in the electronics module 1350 and/or the microneedle array unit 1310, one or more of the following may be included: an analog front end 1502, peripheral sensors 1504 (including one or more of, for example, a thermistor, a real time clock, an ambient light sensor, and a kinetic sensor), a microcontroller 1506, communication circuitry (including in some implementations first wireless communication circuitry 1508 in the electronics module 1350 and second wireless communication circuitry 1510 in the microneedle array unit 1310), one or more power sources (including in some implementations a battery 1512 in the electronics module 1350 and a second battery 1514 in the microneedle array unit 1310), and the output device 1360 (including aspects described above with reference to FIG. 13A-FIG. 13D).

In some variations, fewer, additional, and/or alternative components may be included in one or more of the electronics module 1350 and the microneedle array unit 1310. For example, a voltage regulator, a boost circuit, and other circuitry for processing and/or routing signals may be included in any combination. While FIG. 15A-FIG. 15F depict arrangements and distribution of components between the electronics module 1350 and the microneedle array unit 1310, these depictions are not exhaustive and other allocation of components may be provided.

FIG. 15A depicts a block diagram representation of the wearable analyte monitoring device 1300 including the microneedle array unit 1310 and the electronics module 1350 according to one variation. As shown in FIG. 15A, the electronics module 1350 includes the microcontroller 1506, the peripheral sensors 1504, the first wireless communication circuitry 1508, the battery 1512, and the output device 1360. The microneedle array unit 1310 includes the microneedle array 1340, the analog front end 1502, and the second wireless communication circuitry 1510. In this implementation, the components of the microneedle array unit 1310 provide for analyte measuring and some processing (e.g., the microneedle array 1340 and the analog front end 1502) and communication via the second wireless communication circuitry 1510 to the electronics module 1350. The power source (e.g., the battery 1512) is included in the electronics module 1350, and the electronics module 1350 wirelessly provides power to the microneedle array unit 1310. Data is communicated wirelessly between the two modules.

The analog front end 1502 is capable of implementing analog front end aspects as further described herein and may also include a microcontroller (e.g., a processor) for some signal processing and control aspects. In some variations the functionality of a microcontroller and that of an analog front end are combined in one chip, which may be an application-specific integrated circuit (ASIC), such as a specialized or a customized ASIC. In some variations, a separate microcontroller and a separate analog front end may be incorporated in one of the microneedle array unit 1310 and the electronics module 1350 and may communicate to each other to implement the microcontroller and the analog front end functionality described herein.

The analog front end 1502 may include sensor circuitry that converts analog current measurements from the microneedle array 1340 to digital values for further processing. The analog front end 1502 may, for example, include a programmable analog front end that is suitable for use with electrochemical sensors. In some variations, the analog front end 1502 may be an ultra-low power programmable analog front end for use with electrochemical sensors. In some variations, the analog front end 1502 may be a high precision, impedance, and electrochemical front end. In some variations, the analog front end 1502 may be a configurable analog front end potentiostat for low-power chemical sensing applications. The analog front end 1502 may provide biasing and a complete measurement path, including the analog to digital converters (ADCs). Ultra-low power may allow for the continuous biasing of the microneedle array 1340 to maintain accuracy and fast response.

In some variations, the analog front end 1502 may be compatible with both two and three terminal electrochemical sensors, such as to enable both DC current measurement and AC current measurement capabilities. Furthermore, the analog front end 1502 may include an internal temperature sensor and programmable voltage reference, support external temperature monitoring, provide an external reference source, and integrate voltage monitoring of bias and supply voltages for safety and compliance. In some variations, the analog front end 1502 may include a multi-channel potentiostat to multiplex sensor inputs and handle multiple signal channels.

The microcontroller 1506 and/or the analog front end 1502 may include, for example, a processor with integrated flash memory. In some variations, the microcontroller 1506 and/or the analog front end 1502 may be configured to perform analysis to correlate sensor signals to an analyte measurement (e.g., glucose measurement). For example, the microcontroller 1506 and/or the analog front end 1502 may execute a programmed routine in firmware to interpret the digital signal (e.g., from the analog front end), perform any relevant algorithms and/or other analysis, and route processed data to and/or from a communication module (e.g., the wireless communication circuitry or other communication module).

In some variations, the processing of the sensor signals is split between the microcontroller 1506 of the electronics module 1350 and the analog front end 1502 which may be part of the microneedle array unit 1310 (e.g., as shown in FIG. 15A) or part of the electronics module 1350. The microcontroller 1506 may handle a more significant load of processing compared to that of the analog front end 1502. For example, the analog front end 1502 may convert the analog current measurements from the microneedle array 1340 to digital values and provide the digital values for further processing to the microcontroller 1506. In some variations, the processing of the analog current measurements is split between the microcontroller 1506 and the analog front end 1502 such that additional processing is not needed at other devices (e.g., remote computing devices such as a server, a personal computer, a smartphone, or a smartwatch).

In some variations, the microcontroller 1506 and/or the analog front end 1502 may be configured to activate and/or deactivate analyte sensing operations in response to one or more detected conditions or states of one or more of the environment (e.g., a surrounding area of the microneedle array unit 1310 and/or the electronics module 1350) or components of the microneedle array unit 1310 and/or the electronics module 1350. For example, the microcontroller 1506 and/or the analog front end 1502 may be configured to power on the wearable analyte monitoring device 1300 in response to one or more conditions, such as insertion of the microneedle array 1340 into skin, transition of the wearable analyte monitoring device 1300 from an unusable state to a usable state, connection of the electronics module 1350 and the microneedle array unit 1310, and a command from an external device. The microcontroller 1506 and/or the analog front end 1502 may be configured to power on the wearable analyte monitoring device 1300 in response to a determination of a valid power-on event. Based on the type of valid power-on event, the microcontroller 1506 and/or the analog front end 1502 may transition the wearable analyte monitoring device 1300 to a corresponding mode of operation.

In some variations, the microcontroller 1506 and/or the analog front end 1502 may utilize an 8-bit, 16-bit, 32-bit, or 64-bit data structure. Suitable microcontroller architectures include Reduced Instruction Set Computer (RISC) architectures or Complex Instruction Set Computer (CISC) architectures, and flash memory may be embedded or external to the microcontroller 1506 and/or the analog front end 1502 for suitable data storage. In some variations, the microcontroller 1506 and/or the analog front end 1502 may be a single core microcontroller, while in some variations the microcontroller 1506 and/or the analog front end 1502 may be a multi-core (e.g., dual core) microcontroller which may enable flexible architectures for optimizing power and/or performance within the wearable analyte monitoring device 1300. For example, the cores in the microcontroller 1506 and/or the analog front end 1502 may include similar or differing architectures. For example, in an example variation, the microcontroller 1506 and/or the analog front end 1502 may be a dual core microcontroller including a first core with a high performance and high-power architecture, and a second core with a low performance and low power architecture. The first core may function as a “workhorse” in that it may be used to process higher performance functions (e.g., sensor measurements, algorithmic calculations, etc.), while the second core may be used to perform lower performance functions (e.g., background routines, data transmission, etc.). Accordingly, the different cores of the microcontroller 1506 and/or the analog front end 1502 may be run at different duty cycles (e.g., the second core for lower performance functions may be run at a higher duty cycles) optimized for their respective functions, thereby improving overall power efficiency. In some variations, the microcontroller 1506 and/or the analog front end 1502 may include embedded analog circuitry, such as for interfacing with additional sensors and/or the microneedle array 1340. In some variations, the microcontroller 1506 and/or the analog front end 1502 may be configured to operate using a 0.8V to 5V power source, such as a 1.2V to 3V power source.

With continued reference to FIG. 15A, wireless communication circuitry is included in both the electronics module 1350 and the microneedle array unit 1310 of the wearable analyte monitoring device 1300. The wireless communication circuitry (e.g., the first wireless communication circuitry 1508 and the second wireless communication circuitry 1510) allows for exchange of data and power between the electronics module 1350 and the microneedle array unit 1310. In some variations, the wireless communication circuitry may include a wireless transceiver that is integrated into the microcontroller (e.g., the microcontroller 1506 and/or the analog front end 1502), while in other variations, the wireless transceiver is a separate component. In some variations, the wireless communication circuitry may communicate via wireless network (e.g., through Bluetooth, NFC, WiFi, RFID, Thread, 6LoWPAN, LoRa, or any type of data transmission that is not connected by cables). For example, devices may directly communicate with each other in pairwise connection (1: 1 relationship, e.g., unicasting) or in a hub-spoke or broadcasting connection (“one to many” or 1:m relationship, e.g., multicasting). As another example, the devices may communicate with each other through mesh networking connections (e.g., “many to many”, or m:m relationships, or ad-hoc), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks or use a mix of cellular, Wi-Fi, and satellite communication. In an example variation, the communication module may include a wireless transceiver integrated into the microcontroller and including a Bluetooth Low Energy compatible radio that complies with the Bluetooth Special Interest Group 5.0 specification.

The wireless communication circuitry may further include or be coupled to one or more antennae. For example, the electronics module 1350 may include a chip antenna mounted on the PCB, an antenna implemented directly onto the PCB, or etched or otherwise provided on a surface of the electronics module 1350. Similarly, the microneedle array unit 1310 may include a chip antenna mounted on the PCB, an antenna implemented directly onto the PCB, or etched or otherwise provided on a surface of the microneedle array unit 1310. In a variation, rather than occupy space on the PCB, the antenna may be contained in or etched on the housing, such as on an underside area or topside area of a housing of the electronics module 1350 and/or a surface of the microneedle array unit 1310. For example, a portion of the housing may be metallicized and metal may be deposited to form the antenna, with contacts between the metal and the PCB incorporated. In other variations, a flexible PCB may be incorporated for the antenna and fitted, for example, within the housing cover, and contacts between the flexible PCB and the main PCB may be incorporated. By incorporating the antenna into a space separate from the PCB, additional space is made available on the PCB and increased performance may be achieved by optimizing the placement of the antenna. In a variation, the antenna of the electronics module 1350 and the antenna of the microneedle array unit 1310 are in close proximity to one another when the electronics module 1350 and the microneedle array unit 1310 are coupled. For example, the antennae may be concentric with respect to one another such that coils of the antenna of the electronics module 1350 are adjacent to coils of the antenna of the microneedle array unit 1310 when the electronics module 1350 is engaged within the cavity 1320 of the microneedle array unit 1310.

The first wireless communication circuitry 1508 of the electronics module 1350 may communicate with the microneedle array unit 1310 via a first wireless communication protocol and with external, remote devices via a second wireless communication protocol. For example, the first wireless communication circuitry 1508 may include two or more wireless modules and antennae to enable the first wireless communication protocol and the second wireless communication protocol. In some variations, the first wireless communication protocol is NFC and the second wireless communication protocol is Bluetooth or Bluetooth Low Energy. In some variations, other communication protocols may be used. In a variation, the first wireless communication circuitry 1508 includes an NFC tag IC or NFC module, and the second wireless communication circuitry includes an NFC tag IC or NFC module.

The two or more antennae of the electronics module 1350 may be positioned according to the device or component to which communication and/or power will be directed and/or received. For example, a first antenna of the first wireless communication circuitry 1508 may be an NFC antenna for transmitting data and power to the microneedle array unit 1310 and as such is positioned in an area adjacent to or in contact with the antenna of the microneedle array unit 1310. The second antenna of the first wireless communication circuitry 1508 may be a BLE antenna for transmitting and receiving data from one or more remote devices (such as a smartphone or a smartwatch) and as such is positioned at or near a top, outward facing surface of the electronics module 1350.

In some variations, remote devices can come in and out of range from the first wireless communication circuitry 1508 to connect and reconnect so that a user is able to seamlessly connect and transfer information between devices (e.g., between the wearable analyte monitoring device 1300 and one or more remote devices). In some variations, the microcontroller 1506 and/or the analog front end 1502 may have a unique serial number, enabling tracking of the analyte monitoring device 1300 during production and/or field use.

In some variations, the wearable analyte monitoring device 1300 may be paired to at least one peripheral device such that the peripheral device receives broadcasted or otherwise transmitted data from the wearable analyte monitoring device 1300, including measurement data. Suitable peripheral devices include, for example a mobile computing device (e.g., smartphone, smartwatch) which may be executing a mobile application.

The pairing may be accomplished through suitable wireless communication modules (e.g., NFC and/or Bluetooth). In some variations, the pairing may occur after the wearable analyte monitoring device 1300 is applied and inserted into the skin of a user (e.g., after the wearable analyte monitoring device 1300 is activated). The pairing may occur prior to the microneedle array 1340 being inserted into the skin of a user and/or prior to the coupling of the electronics module 1350 and the microneedle array unit 1310.

Thus, the paired mobile or other device may receive the broadcasted or transmitted data from the wearable analyte monitoring device 1300. The peripheral device may display, store, and/or transmit the measurement data to the user and/or a healthcare provider and/or a support network. Furthermore, in some variations, the paired mobile or wearable device may perform algorithmic treatment to the data to improve the signal fidelity, accuracy, and/or calibration, etc. In some variations, measurement data and/or other user information may additionally or alternatively be communicated and/or stored via a network (e.g., a cloud network).

By way of illustration, in some variations, a mobile computing device or other computing device (e.g., smartphones, smartwatches, tablets, etc.) may be configured to execute a mobile application that provides an interface to display estimated glucose values, trend information, and historical data, etc. Although the below description refers specifically to glucose as a target analyte, the features and processes described below with respect to glucose may be similarly applied to applications relating to other kinds of analytes.

In some variations, the mobile application may use the mobile computing device's wireless communication framework to scan for the wearable analyte monitoring device 1300. The wearable analyte monitoring device 1300 may power on or initialize once it is applied to the skin and/or when the electronics module 1350 is coupled with the microneedle array unit 1310, and the wearable analyte monitoring device 1300 may begin an advertising process. The mobile application may then connect to the wearable analyte monitoring device 1300 and begin priming the microneedle array 1340 for measurement. In case the mobile application detects multiple analyte monitoring devices, the mobile application may detect the wearable analyte monitoring device 1300 that is closest in proximity to itself, may request the user (e.g., via the user interface on the mobile device) to confirm disambiguation, and/or may request a confirmation via a physical interaction with the wearable analyte monitoring device 1300 that is intended for use (e.g., tapping or other prescribed action with the wearable analyte monitoring device 1300 by the user). In some variations, the mobile application may also be capable of connecting to multiple analyte monitoring devices simultaneously. This may be useful, for example, to replace sensors that are reaching the end of their lifetime.

In some variations, the Bluetooth® Low Energy™ (BLE) protocol may be used for connectivity. For example, the sensor implements a custom BLE peripheral profile for the analyte monitoring system. Data may be exchanged after establishing a standard secure BLE connection between the analyte monitoring device and the smartphone, smartwatch, or tablet running the mobile application. The BLE connection may be maintained permanently for the life of the sensor. If the connection is broken due to any reasons (e.g., weak signal) the analyte monitoring device may start advertising itself again and the mobile application may re-establish the connection at the earliest opportunity (e.g., when in range/physical proximity).

In some variations, there may be one or more additional layers of security implemented on top of the BLE connection to ensure authorized access consisting of a combination of one or more techniques such as passcode-protection, shared-secrets, encryption, and multi-factor authentication.

The mobile application may guide the user through initiating a new analyte monitoring device. Once this process completes, the mobile application is not be required for the wearable analyte monitoring device 1300 to operate and record measurements. In some variations, a smart insulin delivery device that is connected to the wearable analyte monitoring device 1300 can be authorized from the mobile application to receive glucose readings from the sensor directly. In some variations, a secondary display device like a smartwatch can be authorized from the mobile application to receive glucose readings from the sensor directly.

Furthermore, in some variations the mobile application may additionally or alternatively help calibrate the wearable analyte monitoring device 1300. For example, the wearable analyte monitoring device 1300 may indicate a request for calibration to the mobile application, and the mobile application may request calibration input from the user to calibrate the sensor.

Implementations of the current subject matter incorporate energy harvesting provided by NFC technology to provide power from the electronics module 1350 to the microneedle array unit 1310. In the presence of the communication field generated by the first wireless communication circuitry 1508, the communication field provides energy to the second wireless communication circuitry 1510. The transferred energy is used by the analog front end 1502 for power-on and processing operations. When the communication field is removed from the microneedle array unit 1310, the analog front end 1502 may return to a shut-down mode until again powered-on by the energy provided by the communication field generated by the first wireless communication circuitry 1508.

With continued reference to FIG. 15A, the electronics module 1350 of the wearable analyte monitoring device 1300 includes a battery 1512 as a power source configured to provide power to the components of the electronics module 1350. The battery 1512 may be any suitable type of battery able to provide power to the various components of the electronics module 1350. The battery 1512 may be a silver-oxide battery, which has a high energy density and is more environmentally friendly than lithium batteries. In some variations, a primary (e.g., non-rechargeable) battery may be used. Furthermore, in some variations, a secondary (e.g., rechargeable) battery may be used. However, any suitable power source may be used, including a rechargeable battery and/or a lithium-based battery. In the variation of the wearable analyte monitoring device 1300 shown in FIG. 15A, and as described above, the microneedle array unit 1310 does not have a battery. Rather, the microneedle array unit 1310 is powered through NFC by the electronics module 1350.

In a variation, the wearable analyte monitoring device 1300 may include one or more sensors in addition to the microneedle array 1340. The one or more sensors (the peripheral sensors 1504) may be included in the electronics module 1350, as shown in FIG. 15A. In a variation, one or more sensors of the peripheral sensors 1504 may be incorporated in the microneedle array unit 1310. One or more temperature sensors may be included and configured to measure skin temperature, which may be used to enable temperature compensation for the microneedle array 1340. For example, in some variations, a thermistor (or other temperature sensor such as a resistance temperature detector, a semiconductor junction, a bimetallic sensor, and a thermopile sensor) may be included and may be arranged near a skin-facing portion or outer facing side of the wearable analyte monitoring device 1300.

A sensor may be incorporated to enable dynamic adjustment of light levels in indicator lights, such as light emitting diodes (LEDs), to compensate for environmental light conditions and to help conserve power.

A kinetic sensor may be used to determine appropriate periods for the wearable analyte monitoring device 1300 to transition to a power saving mode or a reduced power state or to track movement of the user. For example, detection of darkness via the ambient light sensor and no motion via the kinetic sensor may indicate that the wearer of the wearable analyte monitoring device 1300 is asleep or in a relaxed state, which may trigger the wearable analyte monitoring device 1300 to transition to a power saving mode or a reduced power state. The kinetic sensor may, for example, include an accelerometer, a gyroscope, and/or an inertial measurement unit to capture positional, displacement, trajectory, velocity, acceleration, and/or device orientation values. For example, such measurements may be used to infer the wearer's physical activity (e.g., steps, intense exercise) over a finite duration. In some variations, the kinetic sensor may be employed to enable detection of wearer interactions with the wearable analyte monitoring device 1300, such as touch or tapping. For example, touch or tap detection can be employed to silence or snooze notifications, alerts, and alarms, control a wirelessly connected mobile computing device, or to activate and/or deactivate a user interface on the wearable analyte monitoring device 1300 (e.g., an embedded display or indicator light such as the LEDs). Touching or tapping may be performed in a defined sequence and/or for a predetermined duration (e.g., at least 3 seconds, at least 5 seconds) to elicit certain actions (e.g., display or indicator light deactivation and/or activation). In some variations, the wearable analyte monitoring device 1300 may transition to a power saving mode upon detection of limited motion or activity (e.g., absence of significant acceleration) for at least a predetermined period of time (e.g., 15 minutes, 30 minutes, 45 minutes, 1 hour, or other suitable of time), as measured by the kinetic sensor and/or other sensors.

In some variations, the wearable analyte monitoring device 1300 may include at least one real-time clock (RTC). In some variations, the real-time clock has an embedded quartz crystal or the like for maintaining an accurate tracking of time. The real-time clock may be employed to track absolute time (e.g., Coordinated Universal Time, UTC, or local time) when the wearable analyte monitoring device 1300 is in storage or during use. In some variations, synchronization to absolute time may be performed following manufacturing of the wearable analyte monitoring device 1300. During operation, the real-time clock may output a clocking signal to the microcontroller 1506 and/or the analog front end 1502 to drive and/or adjust internal clocks to ensure proper tracking of time. In some variations, the clocking signal from the real-time clock is a constant signal. In some variations, the clocking signal is sent periodically at predefined intervals.

The real-time clock may be employed to time-stamp analyte measurements (e.g., glucose measurements) during operation of the wearable analyte monitoring device 1300 to create a time-series data set that is communicated to a connected peripheral device (e.g., mobile computing device), cloud storage, or other suitable data storage device, such as for later review by the user (e.g., wearer of the analyte monitoring device), a support network, a healthcare provider, etc. In some variations, the microcontroller 1508 and/or the analog front end 1502 performs the time-stamping operations.

The output device 1360, which may be part of the electronics module 1350, may include one or more LEDs. The LEDs may be controlled in one or more predetermined illumination patterns or modes to communicate different statuses and/or other suitable information. An indicator light may be controlled to illuminate with multiple colors (e.g., red, orange, yellow, green, blue, and/or purple, etc.) or in only one color. For example, an indicator light may include a multi-colored LED. As another example, an indicator light may include a transparent or semi-transparent material (e.g., acrylic) positioned over one or more different-colored light sources (e.g., LED) such that different-colored light sources may be selectively activated to illuminate the indicator light in a selected color. The activation of light sources can either occur simultaneously or in sequence. An indicator light may have any suitable form (e.g., raised, flush, recessed, etc. from housing body) and/or shape (e.g., circle or other polygon, ring, elongated strip, etc.). In some variations, an indicator light may have a pinhole size and/or shape to present the same intensity of the light as a larger light source, but with significantly less power requirements, which may help conserve onboard power in the wearable analyte monitoring device 1300.

In some variations, other types of indicator lights may be incorporated in the output device 1360. For example, the indicator lights may include LEDs, OLEDs, lasers, electroluminescent material, or other suitable light sources or waveguides. In some variations, rather than include LEDs or indicator lights, a liquid crystal display (LCD) or an E-ink display may be incorporated.

FIG. 15B depicts a block diagram representation of the wearable analyte monitoring device 1300 including the microneedle array unit 1310 and the electronics module 1350 according to another variation. As shown in FIG. 15B, the electronics module 1350 includes the microcontroller 1506, the peripheral sensors 1504, the first wireless communication circuitry 1508, the battery 1512, and the output device 1360. The electronics module 1350 also includes first electrical contacts 1520 that connect to second electrical contacts 1522 of the microneedle array unit 1310. The microneedle array unit 1310 also includes the microneedle array 1340, the analog front end 1502, and the second wireless communication circuitry 1510. In this implementation, the components of the microneedle array unit 1310 provide for analyte measuring (e.g., the microneedle array 1340 and the analog front end 1502) and communication to the electronics module 1520 via the second wireless communication circuitry 1510 (e.g., data is communicated wirelessly between the two modules). Power, however, is exchanged from the battery 1512 of the electronics module 1350 to the microneedle array unit 1310 through the electrical contacts (e.g., the first electrical contacts 1520 and the second electrical contacts 1522). The electrical contacts may be of the form of the connector pins 1364 and the contact pads 1330 described above with respect to FIG. 13A-FIG. 13D and FIG. 14A-FIG. 14B. However, the electrical contacts may take various other forms to provide connection between the electronics module 1350 and the microneedle array unit 1310 to provide transfer of power to the microneedle array unit 1310.

FIG. 15C depicts a block diagram representation of the wearable analyte monitoring device 1300 including the microneedle array unit 1310 and the electronics module 1350 according to another variation. As shown in FIG. 15C, the electronics module 1350 includes the microcontroller 1506, the peripheral sensors 1504, the first wireless communication circuitry 1508, the output device 1360, and the first electrical contacts 1520. The microneedle array unit 1310 includes the microneedle array 1340, the analog front end 1502, the second wireless communication circuitry 1510, and the second electrical contacts 1522. In this implementation, data is communicated wirelessly between the first communication circuitry 1508 and the second wireless communication circuitry 1510. Power, however, is provided by a second battery 1514 in the microneedle array unit 1310. This is, in the implementation shown in FIG. 15C, the microneedle array unit 1310 powers the electronics module 1350 by transferring power from the second battery 1514 via the second electrical contacts 1522 and the first electrical contacts 1520.

FIG. 15D depicts a block diagram representation of the wearable analyte monitoring device 1300 including the microneedle array unit 1310 and the electronics module 1350 according to another variation. As shown in FIG. 15D, each of the electronics module 1350 and the microneedle array unit 1310 have its own power source, respectively the battery 1512 and the second battery 1514. There is thus not a need to incorporate electrical contacts to transfer power between the microneedle array unit 1310 and the electronics module 1350. However, in some variations, in addition to having dedicated power sources, electrical contacts may be incorporated to transfer power in the event of one power source experiencing a failure.

As also shown in FIG. 15D, the electronics module 1350 includes the microcontroller 1506, the peripheral sensors 1504, the first wireless communication circuitry 1508, and the output device 1360. The microneedle array unit 1310 includes the microneedle array 1340, the analog front end 1502, and the second wireless communication circuitry 1510. In this implementation, the components of the microneedle array unit 1310 provide for analyte measuring (e.g., the microneedle array 1340 and the analog front end 1502) and communication via the second wireless communication circuitry 1510 to the electronics module 1350.

FIG. 15E depicts a block diagram representation of the wearable analyte monitoring device 1300 including the microneedle array unit 1310 and the electronics module 1350 according to another variation. As shown in FIG. 15E, the electronics module 1350 includes the microcontroller 1506, the peripheral sensors 1504, the first wireless communication circuitry 1508, the first battery 1512, and the output device 1360. The electronics module 1350 also includes first electrical contacts 1520 that connect to second electrical contacts 1522 of the microneedle array unit 1310. The microneedle array unit 1310 also includes the microneedle array 1340, and the analog front end 1502. In this implementation, the components of the microneedle array unit 1310 provide for analyte measuring (e.g., the microneedle array 1340 and the analog front end 1502) and communication to the electronics module 1520 via the connection between the second electrical contacts 1522 and the first electrical contacts 1520 (e.g., data is communicated through a wired connection between the two modules). Power is exchanged from the battery 1512 of the electronics module 1350 to the microneedle array unit 1310 through the electrical contacts (e.g., the first electrical contacts 1520 and the second electrical contacts 1522). The electrical contacts may be of the form of the connector pins 1364 and the contact pads 1330 described above with respect to FIG. 13A-FIG. 13D and FIG. 14A-FIG. 14B. However, the electrical contacts may take various other forms to provide connection between the electronics module 1350 and the microneedle array unit 1310 to provide transfer of power and data to the microneedle array unit 1310. In some variations, the microneedle array unit 1310 may include a dedicated power source. The electronics module 1350 includes the first wireless communication circuitry 1508 to provide for wireless communication between other remote devices (e.g., user devices).

FIG. 15F depicts a block diagram representation of the wearable analyte monitoring device 1300 including the microneedle array unit 1310 and the electronics module 1350 according to another variation. As shown in FIG. 15F, the electronics module 1350 includes the analog front end 1502, the microcontroller 1506, the peripheral sensors 1504, the first wireless communication circuitry 1508, the battery 1512, and the output device 1360. The electronics module 1350 also includes first electrical contacts 1520 that connect to second electrical contacts 1522 of the microneedle array unit 1310. The microneedle array unit 1310 also includes the microneedle array 1340. In this implementation, the components of the microneedle array unit 1310 provide for analyte sensing (e.g., the microneedle array 1340 obtains the analyte signals) and communication to the electronics module 1520 via the connection between the second electrical contacts 1522 and the first electrical contacts 1520 (e.g., data is communicated through a wired connection between the two modules). Power is exchanged from the battery 1512 of the electronics module 1350 to the microneedle array unit 1310 through the electrical contacts (e.g., the first electrical contacts 1520 and the second electrical contacts 1522), although in some variations the microneedle array unit 1310 may have its own, dedicated power source. The electrical contacts may be of the form of the connector pins 1364 and the contact pads 1330 described above with respect to FIG. 13A FIG. 13D and FIG. 14A-FIG. 14B. However, the electrical contacts may take various other forms to provide connection between the electronics module 1350 and the microneedle array unit 1310 to provide transfer of power and data to the microneedle array unit 1310. The electronics module 1350 includes the first wireless communication circuitry 1508 to provide for wireless communication between other remote devices (e.g., user devices).

As shown in FIG. 15A-FIG. 15F, batteries (e.g., the battery 1512 and the second battery 1514), wireless communication circuitry (e.g., the first wireless communication circuitry 1508 and the second wireless communication circuitry 1510), and electrical contacts (e.g., the first electrical contacts 1520 and the second electrical contacts 1522) are incorporated in the electronics module 1350 and the microneedle array unit 1310 in various combinations to provide for data and power transfer between the two modules. Any combination of data and power transfer described herein may be incorporated into the electronics module 1350 and the microneedle array unit 1310. In some variations, more than one type of data transfer functionality and/or power transfer functionality may be incorporated.

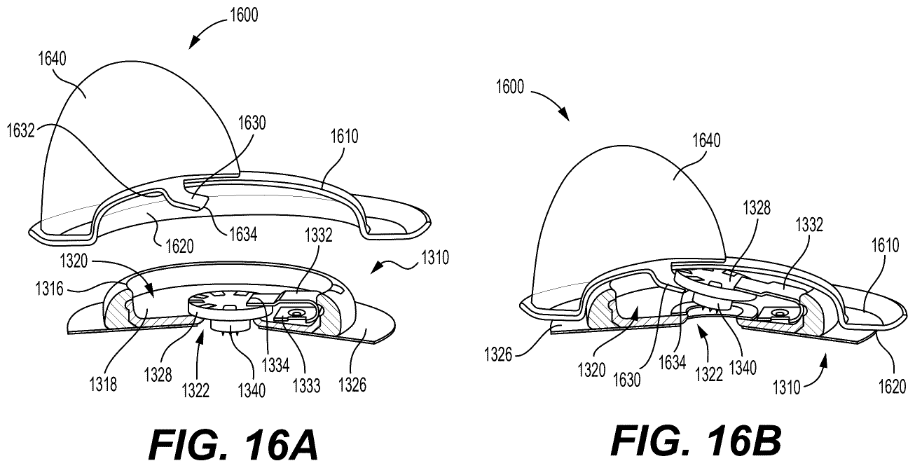

FIG. 16A depicts a perspective side view of a cover 1600 separated from the microneedle array unit 1310, and FIG. 16B depicts a perspective side view of the cover 1600 connected to the microneedle array unit 1310. The cover 1600 is provided to engage with and at least partially surround a proximal, outwardly exposed surface of the microneedle array unit 1310 during storage and transport and also includes features that provide for insertion of the microneedles of the microneedle array 1340 into the skin of a user for analyte sensing. For example, the cover 1600 includes features that engage with and hold the movable retention arm 1332 (and the microneedle array 1340 due to its coupling with the movable retention arm 1332) in an extended configuration. Removal of the cover 1600 facilitates release of the engagement, which causes the movable retention arm (and the microneedle array 1340) to transition to a released configuration in which the microneedles of the microneedle array 1340 penetrate and are inserted into the skin of the user for analyte sensing in the tissue.

In FIG. 16A, the movable retention arm 1332 is in a released (e.g., relaxed) configuration in which the movable retention arm 1332 is not stretched or extended. In this configuration, the microneedle array 1340 extends through the distal opening 1322 formed through the distal surface 1318 of the microneedle array unit 1310. As shown in FIG. 16A, the movable retention arm 1332 has a fixed end 1333 and a movable end 1334. The fixed end 1333 is coupled within the cavity 1320 of the microneedle array unit 1310. For example, the fixed end 1333 may be secured to the distal surface 1318 (e.g., as shown in FIG. 16A) or an inner sidewall of the cavity 1320. The first PCB 1328, or in some variations, the microneedle array 1340, is secured to the movable end 1334. Thus, the first PCB 1328 and the microneedle array 1340 move with the movable end 1334 of the movable retention arm 1332.

The cover 1600 is sized and shaped to correspond to, engage with, and removably couple with at least a portion of the proximal, outwardly exposed surface of the microneedle array unit 1310. The cover 1600 may include a proximal side 1610, a distal side 1620, and a retaining ledge 1630. A graspable fin 1640 may be provided on a portion of the proximal side 1620 of the cover. The cover 1600 is sized and shaped such that the distal side 1620 aligns with and overlays at least a portion of the proximal side of the microneedle array unit 1310. For example, the distal side 1620 of the cover 1600 may have a curved region in which the base 1312 of the microneedle array unit 1310 snugly fits and a planar region surrounding the perimeter of the curved region, where the planar region generally corresponds to and overlays the adhesive layer 1326. When the cover 1600 is connected to the microneedle array unit 1310, as shown in FIG. 16B, the planar region of the cover 1600 may extend past portions of the adhesive layer 1326. In some variations, the cover 1600 may be sized and shaped to cover a portion of the microneedle array unit 1310. For example, the cover 1600 may provide a protective seal around and cover the proximal opening 1316 of the base of the microneedle array unit 1310.

Application of the cover 1600 to the microneedle array unit 1310 (e.g., during manufacturing) may include a pushing or pressing of the cover 1600 onto the microneedle array unit 1310. In some variations, outer sidewalls of the microneedle array unit 1310 and/or the inner sidewalls of the distal side 1620 of the cover 1600 may include one or more engagement features that ensure a secure fit therebetween. For example, one or more compliant features or surfaces may be provided. The compliant features or surfaces may provide a snap-fit or otherwise secure engagement of the microneedle array unit 1310 within the cover 1600.

The cover 1600 may include other curvatures or features to promote removal of the cover 1600 and/or to facilitate connection between the cover 1600 and the microneedle array unit 1310 and/or the skin surface of the user. For example, outer edges of the cover 1600 may be curved upwards or downwards. Additionally, the proximal side 1610 of the cover 1600 may include the graspable fin 1640 to facilitate removal of the cover 1600, as further described herein. In some variations, removal of the cover 1600 may be achieved by pulling or snapping off the cover 1600 without additional features.

The retaining ledge 1630 is a leaf-like or ledge-like projection that has a fixed end 1632 and a free end 1634. The retaining ledge 1630 extends from the distal side 1620 of the cover 1600 (at the fixed end 1632) into the volume defined by the curved region of the cover 1600. When the cover 1600 is coupled with or connected to the microneedle array unit 1310, the free end 1634 of the retaining ledge 1630 extends into the volume defined by the cavity 1320. The retaining ledge 1630 may have a stepped and/or sloped profile and serves as a support structure that engages with the first PCB 1328 (or the microneedle array 1340). For example, as shown in FIG. 16B, when the cover 1600 is applied to the microneedle array unit 1310, a proximal surface of the retaining ledge 1630 slides beneath and engages a distal surface of the first PCB 1328. The configuration of the retaining ledge 1630 (e.g., length and angle at which it extends) is such that the engagement of the retaining ledge 1630 with the first PCB 1328 causes the movable retention arm 1332 to extend from its released, relaxed configuration to an extended configuration, thereby raising the first PCB 1328, the microneedle array 1340, and the movable end 1334 of the movable retention arm 1332. In particular, the extent of the retaining ledge 1630 into the cavity 1320 is less than the depth of the cavity 1320, and when the retaining ledge 1630 engages the first PCB 1328, the movable retention arm 1332 is thus extended.

The retaining ledge 1630 is sized such that when it engages the movable retention arm 1332 so that the movable retention arm 1332 is in an extended configuration, the microneedle array 1340 may be fully contained within the cavity 1320. A protective barrier (not shown), such as a sterile barrier, lining, or cover, may be positioned on the distal side of microneedle array unit 1310 to keep the microneedles in a sterile environment and protect the adhesive layer 1326. The microneedle array unit 1310 with the cover 1600 connected thereto may stay in this configuration during transport and storage until a user is ready to apply the microneedle array unit 1310 for analyte sensing.

The extended configuration of the movable retention arm 1332 caused by the retaining ledge 1630 holding the first PCB 1328 may be one at which the integrity of the movable retention arm 1332 is not impacted. For example, the extended configuration does not impact the ability of the movable retention arm 1332 to move and stay in its released state upon removal from the retaining ledge 1630. In some variations, the engagement of the retaining ledge 1630 with the first PCB 1328 may cause the movable retention arm 1332 to extend to a partially extended configuration that is between the extended configuration and the released configuration. In some variations, the engagement of the retaining ledge 1630 with the first PCB 1328 may cause the movable retention arm 1332 to extend to a fully extended configuration.

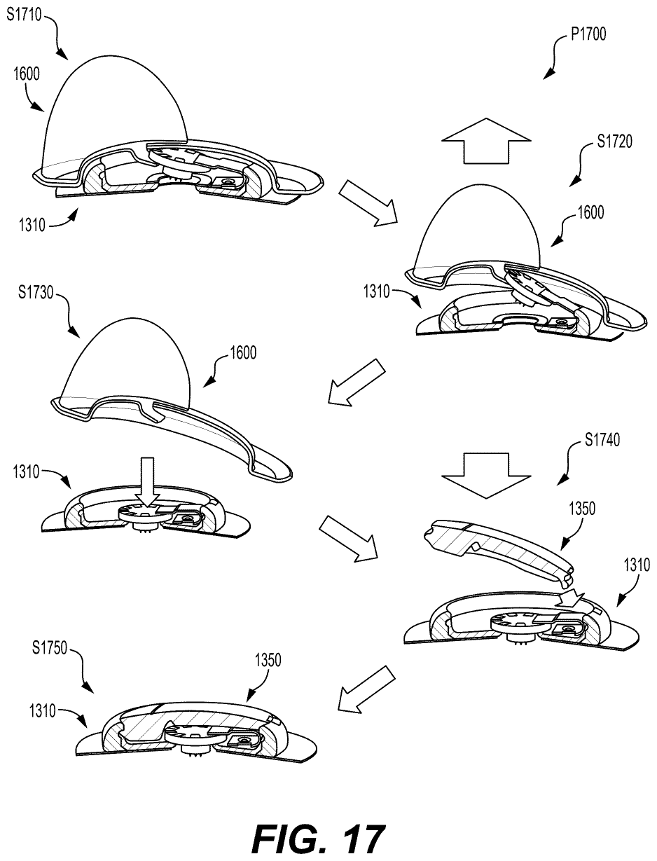

FIG. 17 depicts a variation of a process P1700 of applying the wearable analyte monitoring device 1300. As shown at step S1710, the microneedle array unit 1310 is provided with the cover 1600. The graspable fin 1640 that extends from the proximal side 1610 of the cover 1600 is shaped to allow for a user to easily grasp the cover 1600. For example, the graspable fin 1640 may have a flat and/or contoured area that is sized for a user's fingers to hold on either side to pull the cover 1600 off of the microneedle array unit 1310. The graspable fin 1640 may be replaced with other features that allow the user to pull the cover off of the microneedle array unit 1310, or the cover 1600 may be pulled or snapped or otherwise moved off of the microneedle array unit 1310 (e.g., by grasping outer sidewalls of the cover 1600).

As shown and described herein, the distal side 1620 of the cover 1600 conforms to and aligns with at least a portion of the proximal surface of the microneedle array unit 1310. When the cover 1600 is applied to the microneedle array unit 1310, the retaining ledge 1630 is engaged with the movable retention arm 1332 to move and hold the movable retention arm 1332 in an extended configuration. For example, an engagement may be made between the first PCB 1328 and the retaining ledge 1630 such that the proximal surface of the retaining ledge 1630 (e.g., near the free end 1634) engages with the distal surface of the first PCB 1328 (e.g., the distal surface of the first PCB 1328 rests on the retaining ledge 1630) and extends and holds the movable retention arm 1332 (due to the first PCB 1328 being affixed to the movable retention arm 1332) to an extended configuration. In some configurations, the microneedle array 1340 may rest on the retaining ledge 1630. The movable retention arm 1332 may be moved to and held in an intermediate configuration in which the state of extension is more than that of the released configuration and less than that of the extended configuration.

The user may remove a protective layer or the like from the adhesive layer 1326 and affix the microneedle array unit 1310 to a desired location on the user's skin with the cover 1600 in tact and positioned over the microneedle array unit 1310, as shown at S1710.

At step S1720, the cover 1600 is removed from the microneedle array unit 1310 to release the engagement between the retaining ledge 1630 and the movable retention arm 1332. The release of the engagement causes the movable retention arm 1332 to transition from the extended configuration to a released configuration, thereby snapping the microneedle array 1340 connected at the movable end 1334 of the movable retention arm 1332 such that the microneedles penetrate the skin of the user. The removal of the cover 1600 thus causes insertion of the microneedles of the microneedle array 1340 into skin. The release of the engagement between the retaining ledge 1630 and the movable retention arm 1332 may be caused by pulling the cover 1600 such that the retaining ledge 1630 is pulled past the point of engagement with the first PCB 1328 and/or the microneedle array 1340. The release of the engagement between the retaining ledge 1630 and the movable retention arm 1332 may be caused by sliding the cover 1600 off of the microneedle array unit 1630. In this variation, the retaining ledge 1630 slides away from the point of engagement with the first PCB 1328 and/or the microneedle array 1340. The release of the engagement causes the potential energy stored in the movable retention arm 1332 to be converted into kinetic energy, and the movable retention arm 1332 snaps into the released configuration.

As shown at S1720, as the graspable fin 1640 is pulled to begin removing the cover 1600 from the microneedle array unit 1310, the movable retention arm 1332 is stretched to an extended configuration as the retaining ledge 1630 lifts the first PCB 1328, the microneedle array 1340, and the movable retention arm 1332 in a proximal direction. In the extended configuration, the movable retention arm 1332 may be in a state of extension more than in an intermediate configuration and may be fully or nearly fully extended.

The disengagement between the retaining ledge 1630 and the first PCB 1328 is shown at step S1730. The disengagement releases the first PCB 1328 and causes the movable retention arm 1332 to transition (e.g., snap) from the extended configuration to the released configuration. The release accelerates the first PCB 1328 and the microneedle array 1340 in a distal direction, causing the microneedle array 1340 to extend through the distal opening 1322 of the microneedle array unit 1310 and the microneedles to penetrate the skin of the user.

At step S1740, the electronics module 1350 is aligned within the cavity 1320 of the microneedle array unit 1310. For example, the alignment features may be used to align the electronics module 1350 within the cavity 1320 of the microneedle array unit 1310.

At step S1750, the electronics module 1350 is pushed within the cavity 1320. In variations that include the connector pins 1364 and the contact pads 1330, the coupling of the electronics module 1350 to the microneedle array unit 1310 causes contact to be established between the connector pins 1364 and the contact pads 1330. The wearable analyte monitoring device 1300 is thus assembled and ready for use.

In some variations, the microneedle array unit 1310 may be applied using other application mechanisms. Once applied such that the individual microneedles of the microneedle array 1340 penetrate the skin of the user and are held in place, the electronics module 1350 is inserted and connected to the microneedle array unit 1310. In some variations, the connection of the electronics module 1350 to the microneedle array unit 1310 may assist with maintaining the insertion of the microneedles into the skin of the user.

The wearable analyte monitoring device 1300 according to aspects provided herein is not limited to the exact shapes and forms as those depicted in FIGS. 13-13D, FIGS. 14A-14B, FIGS. 16A-16B, and FIG. 17. For example, and as described above, according to aspects of the current subject matter, certain ones of the electronics components may be in either one or both of the microneedle array unit 1310 and the electronics module 1350. Depending on the distribution of the electronics components between the microneedle array unit 1310 and the electronics module 1350, the shape and the form of each of the microneedle array unit 1310 and the electronics module 1350 may vary.

The wearable analyte monitoring device 1300 may be applied in any suitable location, though in some variations it may be desirable to avoid anatomical areas of thick or calloused skin (e.g., palmar and plantar regions), or areas undergoing significant flexion (e.g., olecranon or patella). Suitable wear sites may include, for example, on the arm (e.g., upper arm, lower arm), shoulder (e.g., over the deltoid), back of hands, neck, face, scalp, torso (e.g., on the back such as in the thoracic region, lumbar region, sacral region, etc. or on the chest or abdomen), buttocks, legs (e.g., upper legs, lower legs, etc.), and/or top of feet.

As described above, the working electrode is the electrode at which the oxidation and/or reduction reaction of interest occurs. In some variations, sensing may be performed at the interface of the working electrode and interstitial fluid located within the body (e.g., on an outer surface of the overall microneedle). In some variations, a working electrode may include an electrode material and a biorecognition layer in which a biorecognition element (e.g., enzyme) is immobilized on the working electrode to facilitate selective analyte quantification. In some variations, the biorecognition layer may also function as an interference-blocking layer and may help prevent endogenous and/or exogenous species from directly oxidizing (or reducing) at the electrode. In some variations, the biorecognition layer and the interference-blocking layer may be separate and distinct layers. In some variations, in addition to the biorecognition layer and/or the combined biorecognition and interference-blocking layer, an electrode protecting layer may be provided for additional protection of the electrode.

In some variations, the working electrode may include an electrode material and a biorecognition layer arranged at least partially over the electrode material, where the biorecognition layer includes an aptamer that selectively and reversibly binds an analyte. The biorecognition layer may include a conductive polymer layer and the aptamer, and the electrode material may include platinum. In some variations, the aptamer may be tethered to the conductive polymer layer via an amide linker. The amide linker may be formed through a reduction of a carboxyl group in the conductive polymer layer and an amine group covalently bound to a 3′ end or a 5′ end of the aptamer, or conversely of an amine group in the conductive polymer layer and a carboxyl group covalently bound to a 3′ end or a 5′ end of the aptamer. In some variations, the electrode material may include gold, and the aptamer may be tethered to the electrode material via a thiol link between the gold and a thiol group covalently bound to a 3′ end or a 5′ end of the aptamer. In some variations, the biorecognition layer may further include 6-mercapto-1-hexanol tethered to the gold via a thiol link. In some variations, the aptamer may be covalently bound to a redox-active molecule at the 3′ end or the 5′ end of the aptamer, such that selective binding of the cortisol to the aptamer and a resulting conformational change of the aptamer brings the redox-active molecule closer to or farther from a surface of the electrode material to facilitate or attenuate electron transfer between the redox-active molecule and the electrode material, thereby generating the sensor signal. In some variations, the redox-active molecule may be methylene blue or an anthraquinone.

Once the analyte monitoring device is inserted and warm-up and any calibration has completed, the analyte monitoring device may be ready for providing sensor measurements of a target analyte. The target analyte (and any requisite co-factor(s)) diffuses from the biological milieu, through the biocompatible and diffusion-limiting layers on the working electrode, and to the biorecognition layer including the biorecognition element. In the presence of a co-factor (if present), the biorecognition element may convert the target analyte to an electroactive product.

A bias potential may be applied between the working and reference electrodes of the analyte monitoring device, and an electrical current may flow from the counter electrode to maintain the fixed potential relationship between the working and reference electrodes. This causes the oxidation or reduction of the electroactive product, causing a current to flow between the working electrodes and counter electrodes. The current value is proportional to the rate of the redox reaction at the working electrode and, specifically, to the concentration of the analyte of interest according to the Cottrell relation.

The electrical current may be converted to a voltage signal by a transimpedance amplifier and quantized to a digital bitstream by means of an analog-to-digital converter (ADC). Alternatively, the electrical current may be directly quantized to a digital bitstream by means of a current-mode ADC. The digital representation of the electrical current may be processed in the embedded microcontroller(s) in the analyte monitoring device and relayed to the wireless communication module for broadcast or transmission (e.g., to one or more peripheral devices). In some variations, the microcontroller may perform additional algorithmic treatment to the data to improve the signal fidelity, accuracy, and/or calibration, etc.

In some variations, the digital representation of the electrical current, or sensor signal, may be correlated to an analyte measurement (e.g., glucose measurement) by the analyte monitoring device. For example, the microcontroller may execute a programmed routine in firmware to interpret the digital signal and perform any relevant algorithms and/or other analysis. Keeping the analysis on-board the analyte monitoring device may, for example, enable the analyte monitoring device to broadcast analyte measurement(s) to multiple devices in parallel, while ensuring that each connected device has the same information. Thus, generally, the user's target analyte (e.g., glucose) values may be estimated and stored in the analyte monitoring device and communicated to one or more peripheral devices.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

### Numbered Embodiments of the Invention

Notwithstanding the appended claims, the disclosure sets forth the following numbered embodiments:
(1) A wearable analyte monitoring device, comprising a microneedle array unit comprising a base comprising a cavity, a movable retention arm coupled to the base, and a microneedle array coupled to the movable retention arm, and an electronics module comprising electronic components within a housing configured to be releasably coupled to the base of the microneedle array unit, wherein the movable retention arm is configured to move between an extended configuration and a released configuration, wherein a plurality of microneedles of the microneedle array extend through a distal opening of the base when the movable retention arm is in the released configuration.
(2) The wearable analyte monitoring device of (1), wherein the electronics housing is configured to fit within the cavity of the base when the movable retention arm is in the released configuration.
(3) The wearable analyte monitoring device of (2), wherein the electronics module wirelessly provides power to the microneedle array unit through a near-field communication field generated by the electronics module, the power received by an antenna of the microneedle array unit.
(4) The wearable analyte monitoring device of either (2) or (3), wherein the microneedle array unit comprises an analog front end configured to receive analyte signals from the microneedle array unit.
(5) The wearable analyte monitoring device of any one of (2) to (4), wherein, when the electronics housing is fitted within the cavity of the base, a plurality of contact pads on a first printed circuit board of the microneedle array unit engages corresponding ones of a plurality of connector pins on a second printed circuit board of the electronics module, wherein one or more of data and power are transmitted through the engagement of the plurality of contact pads and the plurality of connector pins.
(6) The wearable analyte monitoring device of any one of (2) to (5), wherein the electronic components comprise location tracking circuity configured to generate and transmit location data to a remote device in communication with the electronics module.
(7) The wearable analyte monitoring device of either (1) or (2), wherein the movable retention arm comprises a leaf spring.
(8) The wearable analyte monitoring device of any one of (1), (2), or (7), further comprising a cover comprising a proximal side, a distal side, and a retaining ledge coupled to and extending from the distal side, wherein the distal side of the cover is configured to couple with a proximal side of the base, wherein, when the cover is coupled to the base, the retaining ledge engages the movable retention arm such that the engagement causes the movable retention arm to extend to the extended configuration.
(9) The wearable analyte monitoring device of (8), wherein the retaining ledge comprises a fixed end coupled to and extending from the distal side of the cover, and a free end that extends into a volume defined by the distal side of the cover.
(10) The wearable analyte monitoring device of (9), wherein the free end of the retaining ledge engages a distal side of a printed circuit board coupled to the microneedle array.
(11) The wearable analyte monitoring device of either (8) or (9), wherein the distal side of the cover engages outer side walls of the base when the cover is coupled to the base.
(12) The wearable analyte monitoring device of any one of (8), (9), or (11), wherein when the cover is removed from the base, the movable retention arm moves from the extended configuration to the released configuration.
(13) The wearable analyte monitoring device of (12), wherein movement of the movable retention arm from the extended configuration to the released configuration includes transitioning the movable retention arm from a partially extended configuration to the extended configuration.
(14) The wearable analyte monitoring device of either (12) or (13), wherein, when a distal side of the base is positioned on the skin of the user and the movable retention arm is in the released configuration, the plurality of microneedles is arranged within skin of a user.
(15) The wearable analyte monitoring device of any one of (12) to (14), wherein removal of the cover from the base disengages the retaining ledge from the movable retention arm.
(16) The wearable analyte monitoring device of any one of (12) to (15), wherein removal of the cover is caused by a user pulling a graspable fin coupled to the proximal side of the cover.
(17) The wearable analyte monitoring device of any one of (1), (2), (7), or (8), further comprising an adhesive layer coupled to a distal side of the base and surrounding the distal opening.
(18) The wearable analyte monitoring device of any one of (1), (2), (7) (8), or (17), wherein one or more of data and power are transmitted between the microneedle array unit and the electronics module when the housing is fitted within the cavity of the base.
(19) The wearable analyte monitoring device of any one of (1), (2), (7), (8), (17), or (18), further comprising a cover comprising a proximal side, a distal side, and a retaining ledge coupled to and extending from the distal side, wherein the distal side of the cover is configured to couple with a proximal side of the base, wherein, when the cover is coupled to the base, the retaining ledge engages the movable retention arm such that the engagement causes the movable retention arm to extend to a partially extended configuration between the extended configuration and the released configuration.
(20) The wearable analyte monitoring device of (19), wherein the retaining ledge comprises a fixed end coupled to and extending from the distal side of the cover, and a free end that extends into a volume defined by the distal side of the cover.
(21) The wearable analyte monitoring device of (20), wherein the free end of the retaining ledge engages a distal side of a printed circuit board coupled to the microneedle array.
(22) The wearable analyte monitoring device of either (19) or (20), wherein the distal side of the cover engages outer side walls of the base when the cover is coupled to the base.
(23) The wearable analyte monitoring device of any one of (19), (20), or (22), wherein when the cover is lifted away from the base, the movable retention arm moves from the partially extended configuration to the extended configuration.
(24) The wearable analyte monitoring device of (23), wherein when the cover is removed from the base the movable retention arm moves from the extended configuration to the released configuration.
(25) The wearable analyte monitoring device of (24), wherein, when a distal side of the base is positioned on the skin of the user and the movable retention arm is in the release configuration the plurality of microneedles is arranged within skin of a user.
(26) The wearable analyte monitoring device of either (24) or (25), wherein removal of the cover from the base disengages the retaining ledge from the movable retention arm.
(27) The wearable analyte monitoring device of (25), wherein movement of the movable retention arm from the extended configuration to the released configuration includes transitioning the movable retention arm from a partially extended configuration to the extended configuration.
(28) The wearable analyte monitoring device of either (25) or (27), wherein removal of the cover is caused by a user pulling a graspable fin coupled to the proximal side of the cover.
(29) A method of applying a wearable analyte monitoring device, comprising applying a distal side of a microneedle array unit to a skin surface of a user, the microneedle array unit comprising a base comprising a cavity, a movable retention arm coupled within the cavity, and a microneedle array comprising a plurality of microneedles configured to sense an analyte in tissue of the user, the microneedle array coupled to the movable retention arm, applying a force to a cover coupled to the microneedle array unit to transition the movable retention arm from an extended configuration to a released configuration, and inserting an electronics module into the cavity of the base of the microneedle array unit.
(30) The method of (29), wherein one or more of data and power are transmitted between the microneedle array unit and the electronics module when the electronics module is fitted within the cavity of the base.
(31) The method of either (29) or (30), wherein, when the cover is coupled to the base, a retaining ledge extending from a distal side of the cover engages the movable retention arm in the extended configuration.
(32) The method of any one of (29) to (31), wherein applying the force to the cover to transition the movable retention arm from the extended configuration to the released configuration includes transitioning the movable retention arm from a partially extended configuration to the extended configuration.
(33) A wearable analyte monitoring device, comprising a microneedle array unit comprising a base comprising a body, the body comprising side walls, a proximal opening, a distal surface opposite the proximal opening and comprising a distal opening, and a cavity defined by the side walls, the proximal opening, and the distal surface, a movable retention arm coupled within the cavity, and a microneedle array comprising a plurality of microneedles, the microneedle array coupled to the movable retention arm, wherein the movable retention arm is configured to move between an extended configuration and a released configuration, wherein the plurality of microneedles extend through the distal opening when the movable retention arm is in the released configuration, and an electronics module comprising an electronics housing defining an interior, and electronic components arranged within the interior of the electronics housing, wherein the electronics housing body is configured to fit within the cavity of the base body, wherein one or more of data and power are transmitted between the microneedle array unit and the electronics module when the electronics housing body is fitted within the cavity of the base body.
(34) A wearable analyte monitoring device, comprising a microneedle array unit comprising a base comprising a cavity, a movable retention arm coupled within the cavity, and a microneedle array comprising a plurality of microneedles configured to sense an analyte in skin of a user, the microneedle array coupled to the movable retention arm, and an electronics module comprising an electronics housing defining an interior in which electronic components are arranged, the electronics housing configured to releasably fit within the cavity of the base of the microneedle array unit, wherein the movable retention arm is configured to move between an extended configuration and a released configuration, wherein the plurality of microneedles extend through a distal opening of a distal surface of the base when the movable retention arm is in the released configuration, and wherein one or more of data and power are transmitted between the microneedle array unit and the electronics module when the electronics housing is fitted within the cavity of the base.

1. A wearable analyte monitoring device, comprising:
a microneedle array unit comprising a base comprising a cavity, a movable retention arm coupled to the base, and a microneedle array coupled to the movable retention arm; and
an electronics module comprising electronic components within a housing configured to be releasably coupled to the base of the microneedle array unit,
wherein the movable retention arm is configured to move between an extended configuration and a released configuration, wherein a plurality of microneedles of the microneedle array extend through a distal opening of the base when the movable retention arm is in the released configuration.

2. The wearable analyte monitoring device of claim 1, wherein the electronics housing is configured to fit within the cavity of the base when the movable retention arm is in the released configuration.

3-7. (canceled)

8. The wearable analyte monitoring device of claim 1, further comprising a cover comprising a proximal side, a distal side, and a retaining ledge coupled to and extending from the distal side,
wherein the distal side of the cover is configured to couple with a proximal side of the base,
wherein, when the cover is coupled to the base, the retaining ledge engages the movable retention arm such that the engagement causes the movable retention arm to extend to the extended configuration.

9. The wearable analyte monitoring device of claim 8, wherein the retaining ledge comprises a fixed end coupled to and extending from the distal side of the cover, and a free end that extends into a volume defined by the distal side of the cover.

10. The wearable analyte monitoring device of claim 9, wherein the free end of the retaining ledge engages a distal side of a printed circuit board coupled to the microneedle array.

11. The wearable analyte monitoring device of claim 8, wherein the distal side of the cover engages outer side walls of the base when the cover is coupled to the base.

12. The wearable analyte monitoring device of claim 8, wherein when the cover is removed from the base, the movable retention arm moves from the extended configuration to the released configuration.

13. The wearable analyte monitoring device of claim 12, wherein movement of the movable retention arm from the extended configuration to the released configuration includes transitioning the movable retention arm from a partially extended configuration to the extended configuration.

14. (canceled)

15. The wearable analyte monitoring device of claim 12, wherein removal of the cover from the base disengages the retaining ledge from the movable retention arm.

16. (canceled)

17. The wearable analyte monitoring device of claim 1, further comprising an adhesive layer coupled to a distal side of the base and surrounding the distal opening.

18. The wearable analyte monitoring device of claim 1, wherein one or more of data or power are transmitted between the microneedle array unit and the electronics module when the housing is fitted within the cavity of the base.

19. The wearable analyte monitoring device of claim 1, further comprising a cover comprising a proximal side, a distal side, and a retaining ledge coupled to and extending from the distal side,
wherein the distal side of the cover is configured to couple with a proximal side of the base,
wherein, when the cover is coupled to the base, the retaining ledge engages the movable retention arm such that the engagement causes the movable retention arm to extend to a partially extended configuration between the extended configuration and the released configuration.

20. The wearable analyte monitoring device of claim 19, wherein the retaining ledge comprises a fixed end coupled to and extending from the distal side of the cover, and a free end that extends into a volume defined by the distal side of the cover.

21. The wearable analyte monitoring device of claim 20, wherein the free end of the retaining ledge engages a distal side of a printed circuit board coupled to the microneedle array.

22. (canceled)

23. The wearable analyte monitoring device of claim 19, wherein when the cover is lifted away from the base, the movable retention arm moves from the partially extended configuration to the extended configuration.

24. The wearable analyte monitoring device of claim 23, wherein when the cover is removed from the base the movable retention arm moves from the extended configuration to the released configuration.

25. (canceled)

26. The wearable analyte monitoring device of claim 24, wherein removal of the cover from the base disengages the retaining ledge from the movable retention arm.

27. (canceled)

28. (canceled)

29. A method of applying a wearable analyte monitoring device, comprising:
applying a distal side of a microneedle array unit to a skin surface of a user, the microneedle array unit comprising a base comprising a cavity, a movable retention arm coupled within the cavity, and a microneedle array comprising a plurality of microneedles configured to sense an analyte in tissue of the user, the microneedle array coupled to the movable retention arm;
applying a force to a cover coupled to the microneedle array unit to transition the movable retention arm from an extended configuration to a released configuration; and
inserting an electronics module into the cavity of the base of the microneedle array unit.

30. (canceled)

31. The method of claim 29, wherein, when the cover is coupled to the base, a retaining ledge extending from a distal side of the cover engages the movable retention arm in the extended configuration.

32. (canceled)

33. (canceled)

34. A wearable analyte monitoring device, comprising:
a microneedle array unit comprising a base comprising a cavity, a movable retention arm coupled within the cavity, and a microneedle array comprising a plurality of microneedles configured to sense an analyte in skin of a user, the microneedle array coupled to the movable retention arm; and
an electronics module comprising an electronics housing defining an interior in which electronic components are arranged, the electronics housing configured to releasably fit within the cavity of the base of the microneedle array unit,
wherein the movable retention arm is configured to move between an extended configuration and a released configuration, wherein the plurality of microneedles extend through a distal opening of a distal surface of the base when the movable retention arm is in the released configuration, and
wherein one or more of data or power are transmitted between the microneedle array unit and the electronics module when the electronics housing is fitted within the cavity of the base.
